(19) United States
(12) Patent Application Publication
(10) Pub. No.: US 2009/0285750 A1
(43) Pub. Date: Nov. 19, 2009

(54) AGENT, COMPOSITION AND METHOD

(76) Inventors: Carl Borrebaeck, Lund (SE); Johan Fransson, San Diego, CA (US)

Correspondence Address: FISH & RICHARDSON P.C., PO BOX 1022, MINNEAPOLIS, MN, 55440-1022, US

(21) Appl. No.: 12/296,819

(22) PCT Filed: Apr. 11, 2007
(86) PCT No.: PCT/EP2007/003216

§ 371 Date: Feb. 25, 2009

(30) Foreign Application Priority Data
Apr. 12, 2006 (GB) 0607354.8

(51) Int. Cl. A61K 51/10; A61K 38/00; A61K 31/7088; A61K 51/00; A61K 51/08; A61P 35/00; G01N 33/53; C07H 21/00; C12N 15/63; C12N 1/21; C12N 5/10; C12P 1/00

(52) U.S. Cl. 424/1.49; 514/2; 424/1.11; 424/1.61; 424/1.69; 435/7.23; 536/23.1; 435/320.1; 435/252.3; 435/325; 435/41

(57) ABSTRACT

The present application relates to agents, compositions and methods for use in medicine. In particular, the application relates to an agent comprising a binding moiety capable of selectively binding to Ku protein for use in medicine, in particular in the treatment and diagnosis of cancers.

The present invention relates to agents, compositions and methods for use in medicine. In particular, the invention relates to an agent comprising a binding moiety capable of selectively binding to Ku protein for use in medicine, in particular in the treatment of cancers.

To be of therapeutic relevance, a tumor-associated antigen should be expressed on the surface of neoplastic cells but not, or to a significantly lower extent, on cells of non-transformed nature. Cell surface receptors are key targets for antibody-based therapy of cancer. Upon antibody binding, the targeted membrane-associated antigens might relay signalling into the cells e.g. initiation of apoptosis (Shan et al., 1998; Fransson et al., 2006) or be internalized by receptor-mediated endocytosis (Liu et al., 2004; Fransson et al., 2004). Internalization of the receptor after antibody engagement opens a window for a wide range of therapeutic interventions, such as antibody-drug conjugates (Wu, 2005) or antibody mediated gene therapy (Zhang et al., 2002).

The Ku protein is a heterodimer of two tightly associated sub-units called Ku70 and Ku80, normally located in the nucleus, where it is involved in non-homologous end joining processes, which is responsible for repairing DNA double strand breaks (DSB). Ku binds to the damaged DNA and recruits the catalytic sub-unit of the DNA dependent protein kinase (DNA-PKCs), which ultimately leads to engagement of DSB repair enzymes (Lieber et al., 2003). Accordingly, transgenic mice with Ku deletions display a profound defect in cell proliferation and a hypersensitivity against ionizing radiation due to a defect in DSB repair (Lees-Miller and Meek, 2003).

The exclusive nuclear localization of the Ku protein has been reassessed by studies that demonstrate Ku to be expressed on the surface of tumor cell lines. It has been shown to be pluripotent, since its expression in the plasma membrane in transformed cells (Prabhakar et al., 1990) seems to be involved in adhesion, migration and invasion (reviewed in Muller et al., 2005), where the latter activity recently was shown to be mediated by a specific interaction between Ku and metalloproteinase 9 (Monferran et al., 2004). Furthermore, in the cytosol Ku70 interacts with the pro-apoptotic protein Bax and prevents its mitochondrial translocation, suggesting that Ku70 suppresses Bax-mediated apoptosis (Sawada, M. et al., 2003). Thus, Ku seems to be involved in pro-survival as well as pro-invasive processes, both important features of tumor progression (Hanahan and Weinberg, 2000), and has been reported on several human cell lines but, so far only on primary multiple myelomas (Muller et al., 2005; Tai et al., 2002). On human untransformed cells the expression of Ku70 has only been detected on human umbilical vein endothelial cells and monocyte-derived macrophages (Ginis et al., 1995; Monferran et al., 2004).

The present inventors have now surprisingly discovered that the Ku protein is internalised on binding an extracellular agent, such as an antibody. The inventors have shown that receptor-mediated endocytosis of the Ku protein is rapid (t_{1/2} 12 min) and extensive (90% of the receptor pool inside the cell after 100 min) and may be used as a port of entry for cytotoxic payloads to tumor cells of various origin. This surprising discovery suggests that the internalisation properties of the Ku protein may be used as a port of entry for delivering cytotoxic agents to tumour cells and in selective drug-delivery and therapy of cancers.

Accordingly, in a first aspect, the invention provides an agent comprising a binding moiety capable of selectively binding to Ku protein for use in medicine.

By “an agent” we include any purified or isolated natural or chemically-synthesised entity comprising one or more molecule. Preferably, the term includes one or more polynucleotide and/or one or more polypeptide and/or one or more small chemical molecule, wherein said polynucleotide and/or polypeptide and/or small chemical molecule may or may not be modified by the ionic and/or covalent addition of chemical groups.

By “binding moiety” we include a region or regions of the agent of the invention capable of reversibly and/or irreversibly associating with a region or regions of another molecule or molecules by covalent and/or ionic interaction.

By “selectively binding” we include the ability of the agent of the invention to bind at least 10-fold more strongly to the Ku protein defined herein than to another polypeptide; preferably at least 50-fold more strongly and more preferably at least 100-fold more strongly. Preferably, the agent binds to the Ku protein under physiological conditions, for example, in vivo.

In human cells, the Ku protein is a heterodimer comprising a 70 kDa and an 80 kDa subunit termed Ku70 and Ku80, respectively, and is responsible for repairing DNA double strand breaks (DSB). By “Ku protein” we include any natural or synthetic protein comprising one or more of the human monomers and/or homologues and/or orthologues of the human Ku protein monomers and/or proteins with structural and/or functional identity to the human Ku protein as defined herein and/or natural variants thereof.

Preferably, the Ku protein is a human protein, but it may be from any mammal such as a domesticated mammal (preferably of agricultural or commercial significance including a horse, pig, cow, sheep, dog and cat). By “mammalian protein” we include any protein found in, derived from, and/or isolated from, one or more cells of a mammal; for example, the term “human protein” includes a protein found in, derived from, and/or isolated from one or more cells of a human.

Preferably, the invention provides an agent wherein the Ku protein comprises the Ku-70 monomer and/or the Ku-80 monomer. More preferably, the Ku protein is a heterodimer, conveniently a Ku-70/80 heterodimer.

Preferably, the invention provides an agent wherein the Ku70 protein comprises the polypeptide sequence of SEQ ID NO:1 and/or is encoded by the polynucleotide sequence of SEQ ID NO:3 and/or the Ku80 protein comprises the polypeptide sequence of SEQ ID NO:2 and/or is encoded by the polynucleotide sequence of SEQ ID NO:4.

By “natural variants” we include, for example, allelic variants. Typically, these will vary from the given sequence by only one or two or three, and typically no more than 10 or 20 amino acid residues. Typically, the variants have conservative substitutions.

Variants of the polypeptides defined herein include polypeptides comprising a sequence with at least 60% identity to an amino acid sequence selected from the group comprising: SEQ ID NO:1; SEQ ID NO:2; SEQ ID NO:5; SEQ ID NO:6; SEQ ID NO:7; SEQ ID NO:8; SEQ ID NO:9; SEQ ID NO:10 and SEQ ID NO:11; and is preferably at least 70% or 80% or 85% or 90% identity to said sequence, and more preferably at least 95%, 96%, 97%, 98% or 99% identity to said sequence.

Percent identity can be determined by, for example, the LALIGN program (Huang and Miller, Adv. Appl. Math. (1991) 12:337-357) at the Expasy facility site (http://www.ch.embnet.org/software/LALIGN_form.html) using as parameters the global alignment option, scoring matrix BLOSUM62, opening gap penalty −14, extending gap penalty −4. Alternatively, the percent sequence identity between two polypeptides may be determined using suitable computer programs, for example the GAP program of the University of Wisconsin Genetic Computing Group and it will be appreciated that percent identity is calculated in relation to polypeptides whose sequence has been aligned optimally.

Preferably, the invention provides an agent wherein the Ku protein is localised on the surface of a cell and, more preferably, the Ku protein is localised on the surface of a cancer cell.

By “localised on the surface of a cell” we include the meaning that the Ku protein is associated with the cell such that one or more region of the Ku protein is present on outer face of the cell surface. For example, the Ku protein may be inserted into the cell plasma membrane (i.e. orientated as a transmembrane protein) with one or more region presented on the extracellular surface. Alternatively, the entire Ku protein may be outside the cell with covalent and/or ionic interactions localising it to a specific region or regions of the cell surface.

Thus, by “surface of a cancer cell” we include the meaning that the Ku protein is localised in such a manner in relation to one or more cell derived from, or characteristic of, a cancerous cell or tumour.

Preferably, the invention provides an agent capable of inducing and/or increasing intracellular internalisation of the Ku protein and/or complex comprising the agent and Ku protein.

By “intracellular internalisation” we include the molecular, biochemical and cellular events associated with the process of translocating a molecule from the extracellular surface of a cell to the intracellular surface of a cell. The processes responsible for intracellular internalisation of molecules is well-known to those skilled in the field of molecular and cellular biology and can involve the internalisation of extracellular molecules (such as hormones, antibodies, and small organic molecules); membrane-associated molecules (such as cell-surface receptors); and complexes of membrane-associated molecules bound to extracellular molecules (for example, a ligand bound to a transmembrane receptor or an antibody bound to a membrane-associated molecule).

Thus, by “inducing and/or increasing intracellular internalisation” we include events wherein intracellular internalisation is initiated and/or the rate and/or extent of intracellular internalisation is increased.

Preferably, the invention provides an agent further comprising a cytotoxic moiety; more preferably, the cytotoxic moiety is directly and/or indirectly cytotoxic.

By “directly cytotoxic” we include the meaning that the moiety is one which on its own is cytotoxic. By “indirectly cytotoxic” we include the meaning that the moiety is one which, although is not itself cytotoxic, can induce cytotoxicity, for example by its action on a further molecule or by further action on it.

Conveniently, the cytotoxic moiety is cytotoxic when intracellular and, preferably, is not cytotoxic when extracellular.

Preferably, the invention provides an agent wherein the cytotoxic moiety is a directly cytotoxic chemotherapeutic agent. Optionally, the cytotoxic moiety is a directly cytotoxic polypeptide. Cytotoxic chemotherapeutic agents are well known in the art.

Cytotoxic chemotherapeutic agents, such as anticancer agents, include: alkylating agents including nitrogen mustards such as mechlorethamine (HN₂), cyclophosphamide, ifosfamide, melphalan (L-sarcolysin) and chlorambucil; ethylenimines and methylmelamines such as hexamethylmelamine, thiotepa; alkyl sulphonates such as busulfan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); Antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin). Natural Products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes. Miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; and hormone agonists/antagonists such as flutamide and tamoxifen.

Various of these agents have previously been attached to antibodies and other target site-delivery agents, and so agents of the invention comprising these agents may readily be made by the person skilled in the art. For example, carbodiimide conjugation (Bauminger & Wilchek (1980) Methods Enzymol. 70, 151-159; incorporated herein by reference) may be used to conjugate a variety of agents, including doxorubicin, to antibodies or peptides.

Carbodiimides comprise a group of compounds that have the general formula R₁—N═C═N—R₂, where R₁ and R₂ can be aliphatic or aromatic, and are used for synthesis of peptide bonds. The preparative procedure is simple, relatively fast, and is carried out under mild conditions. Carbodiimide compounds attack carboxylic groups to change them into reactive sites for free amino groups.

The water soluble carbodiimide, 1-ethyl-3-(3-dimethylaminopropyl)carbodiimide (EDC) is particularly useful for conjugating a functional moiety to a binding moiety and may be used to conjugate doxorubicin to tumour homing peptides. The conjugation of doxorubicin and a binding moiety requires the presence of an amino group, which is provided by doxorubicin, and a carboxyl group, which is provided by the binding moiety such as an antibody or peptide.

In addition to using carbodiimides for the direct formation of peptide bonds, EDC also can be used to prepare active esters such as N-hydroxysuccinimide (NHS) ester. The NHS ester, which binds only to amino groups, then can be used to induce the formation of an amide bond with the single amino group of the doxorubicin. The use of EDC and NHS in combination is commonly used for conjugation in order to increase yield of conjugate formation (Bauminger & Wilchek, supra, 1980).

Other methods for conjugating a cytotoxic moiety to a binding moiety also can be used. For example, sodium periodate oxidation followed by reductive alkylation of appropriate reactants can be used, as can glutaraldehyde cross-linking. However, it is recognised that, regardless of which method of producing a conjugate of the invention is selected, a determination must be made that the binding moiety maintains its targeting ability and that the functional moiety maintains its relevant function.

In one embodiment of the invention, the cytotoxic moiety is a cytotoxic peptide or polypeptide moiety by which we include any moiety which leads to cell death.

Cytotoxic peptide and polypeptide moieties are well known in the art and include, for example, ricin, abrin, Pseudomonas exotoxin, tissue factor and the like. Methods for linking them to targeting moieties such as antibodies are also known in the art. The use of ricin as a cytotoxic agent is described in Burrows & Thorpe (1993) Proc. Natl. Acad. Sci. USA 90, 8996-9000, incorporated herein by reference, and the use of tissue factor, which leads to localised blood clotting and infarction of a tumour, has been described by Ran et al (1998) Cancer Res. 58, 4646-4653 and Huang et al (1997) Science 275, 547-550. Tsai et al (1995) Dis. Colon Rectum 38, 1067-1074 describes the abrin A chain conjugated to a monoclonal antibody and is incorporated herein by reference. Other ribosome inactivating proteins are described as cytotoxic agents in WO 96/06641. Pseudomonas exotoxin may also be used as the cytotoxic polypeptide moiety (see, for example, Aiello et al (1995) Proc. Natl. Acad. Sci. USA 92, 10457-10461; incorporated herein by reference).

Certain cytokines, such as TNFα and IL-2, may also be useful as cytotoxic agents.

Certain radioactive atoms may also be cytotoxic if delivered in sufficient doses. Thus, the cytotoxic moiety may comprise a radioactive atom which, in use, delivers a sufficient quantity of radioactivity to the target site so as to be cytotoxic. Suitable radioactive atoms include phosphorus-32, iodine-125, iodine-131, indium-111, rhenium-186, rhenium-188 or yttrium-90, or any other isotope which emits enough energy to destroy neighbouring cells, organelles or nucleic acid. Preferably, the isotopes and density of radioactive atoms in the agents of the invention are such that a dose of more than 4000 cGy (preferably at least 6000, 8000 or 10000 cGy) is delivered to the target site and, preferably, to the cells at the target site and their organelles, particularly the nucleus.

The radioactive atom may be attached to the binding moiety in known ways. For example, EDTA or another chelating agent may be attached to the binding moiety and used to attach ¹¹¹In or ⁹⁰Y. Tyrosine residues may be directly labelled with ¹²⁵I or ¹³¹I.

The cytotoxic moiety may be a suitable indirectly-cytotoxic polypeptide. In a particularly preferred embodiment, the indirectly cytotoxic polypeptide is a polypeptide which has enzymatic activity and can convert a non-toxic and/or relatively non-toxic prodrug into a cytotoxic drug. When the binding moiety is an antibody this type of system is often referred to as ADEPT (Antibody-Directed Enzyme Prodrug Therapy). The system requires that the binding moiety locates the enzymatic portion to the desired site in the body of the patient and after allowing time for the enzyme to localise at the site, administering a prodrug which is a substrate for the enzyme, the end product of the catalysis being a cytotoxic compound. The object of the approach is to maximise the concentration of drug at the desired site and to minimise the concentration of drug in normal tissues (see Senter, P. D. et al (1988) “Anti-tumour effects of antibody-alkaline phosphatase conjugates in combination with etoposide phosphate” Proc. Natl. Acad. Sci. USA 85, 4842-4846; Bagshawe (1987) Br. J. Cancer 56, 531-2; and Bagshawe, K. D. et al (1988) “A cytotoxic agent can be generated selectively at cancer sites” Br. J. Cancer. 58, 700-703).

Clearly, any binding moiety with specificity for Ku protein may be used in place of an antibody in this type of directed enzyme prodrug therapy system. For example, scaffold structures that have a high degree of stability yet allow variability to be introduced at certain positions may be used to create molecular libraries from which binding moieties can be derived. The best characterised of these are a fibronectin domain and a 58-amino-acid large protein A domain which tolerate variability (Nygren and Uhlen, Scaffolds for engineering novel binding sites in proteins. Curr Opin Struct Biol. 1997 August; 7 (4):463-9). Another scaffold structure that may be used is a structure based on the fibronectin scaffold which has been used to generate high affinity specific binders to a variety of target molecules (Weng et al. Proteomics, 2001, 2:48-57). There are also other molecular folds that allow some degree of variation. Such examples include major histocompatibility complex (MHC) class I and II molecules and recently a novel class of molecules termed “defensins” have been identified to be similar in basic structure while still harbouring extensive sequence variability in-between the gene family members.

In a preferred embodiment, the invention provides an agent wherein the cytotoxic moiety is capable of converting a non-cytotoxic prodrug into a cytotoxic drug.

The enzyme and prodrug of the system using a targeted enzyme as described herein may be any of those previously proposed. The cytotoxic substance may be any existing anti-cancer drug such as an alkylating agent; an agent which intercalates in DNA; an agent which inhibits any key enzymes such as dihydrofolate reductase, thymidine synthetase, ribonucleotide reductase, nucleoside kinases or topoisomerase; or an agent which effects cell death by interacting with any other cellular constituent. Etoposide is an example of a topoisomerase inhibitor.

Reported prodrug systems include: a phenol mustard prodrug activated by an E. coli β-glucuronidase (Wang et al, 1992 and Roffler et al, 1991); a doxorubicin prodrug activated by a human β-glucuronidase (Bosslet et al, 1994); further doxorubicin prodrugs activated by coffee bean α-galactosidase (Azoulay et al, 1995); daunorubicin prodrugs, activated by coffee bean α-D-galactosidase (Gesson et al, 1994); a 5-fluorouridine prodrug activated by an E. coli β-D-galactosidase (Abraham et al, 1994); and methotrexate prodrugs (e.g. methotrexate-alanine) activated by carboxypeptidase A (Kuefner et al, 1990, Vitols et al, 1992 and Vitols et al, 1995). These and others are included in Table A, below.

TABLE A

| Enzyme | Prodrug |
|---|---|
| Carboxypeptidase G2 | Derivatives of L-glutamic acid and benzoic acid |
| | mustards, aniline mustards, phenol mustards and |
| | phenylenediamine mustards; fluorinated |
| | derivatives of these |
| Alkaline phosphatase | Etoposide phosphate |
| | Mitomycin phosphate |
| Beta-glucuronidase | p-Hydroxyaniline mustard-glucuronide |
| | Epirubicin-glucuronide |
| Penicillin-V-amidase | Adriamycin-N phenoxyacetyl |
| Penicillin-G-amidase | N-(4′-hydroxyphenyl acetyl) palytoxin |
| | Doxorubicin and melphalan |
| Beta-lactamase | Nitrogen mustard-cephalosporin |
| | p-phenylenediamine; doxorubicin derivatives; |
| | vinblastine derivative-cephalosporin, |
| | cephalosporin mustard; a taxol derivative |
| Beta-glucosidase | Cyanophenylmethyl-beta-D-gluco- |
| | pyranosiduronic acid |
| Nitroreductase | 5-(Azaridin-1-yl-)-2,4-dinitrobenzamide |
| Cytosine deaminase | 5-Fluorocytosine |
| Carboxypeptidase A | Methotrexate-alanine |

Table A is adapted from Bagshawe (1995) Drug Dev. Res. 34, 220-230, from which full references for these various systems may be obtained; the taxol derivative is described in Rodrigues, M. L. et al (1995) Chemistry & Biology 2, 223).

Suitable enzymes for forming part of the enzymatic portion a agent of the invention include: exopeptidases, such as carboxypeptidases G, G1 and G2 (for glutamylated mustard prodrugs), carboxypeptidases A and B (for MTX-based prodrugs) and aminopeptidases (for 2-α-aminocyl MTC prodrugs); endopeptidases, such as e.g. thrombolysin (for thrombin prodrugs); hydrolases, such as phosphatases (e.g. alkaline phosphatase) or sulphatases (e.g. aryl sulphatases) (for phosphylated or sulphated prodrugs); amidases, such as penicillin amidases and arylacyl amidase; lactamases, such as β-lactamases; glycosidases, such as β-glucuronidase (for β-glucuronomide anthracyclines), α-galactosidase (for amygdalin) and β-galactosidase (for β-galactose anthracycline); deaminases, such as cytosine deaminase (for 5FC); kinases, such as urokinase and thymidine kinase (for gancyclovir); reductases, such as nitroreductase (for CB1954 and analogues), azoreductase (for azobenzene mustards) and DT-diaphorase (for CB1954); oxidases, such as glucose oxidase (for glucose), xanthine oxidase (for xanthine) and lactoperoxidase; DL-racemases, catalytic antibodies and cyclodextrins.

Preferably, the prodrug is relatively non-toxic compared to the cytotoxic drug. Typically, it has less than 10% of the toxicity, preferably less than 1% of the toxicity as measured in a suitable in vitro cytotoxicity test.

It is likely that the moiety which is able to convert a prodrug to a cytotoxic drug will be active in isolation from the rest of the agent of the invention but it is necessary only for it to be active when (a) it is in combination with the rest of the agent of the invention and (b) the agent of the invention is attached to, adjacent to or internalised in target cells.

When each moiety of the agent of the invention is a polypeptide, the two portions may be linked together by any of the conventional ways of cross-linking polypeptides, such as those generally described in O'Sullivan et al (1979) Anal. Biochem. 100, 100-108. For example, the binding moiety may be enriched with thiol groups and the further moiety reacted with a bifunctional agent capable of reacting with those thiol groups, for example the N-hydroxysuccinimide ester of iodoacetic acid (NHIA) or N-succinimidyl-3-(2-pyridyldithio)propionate (SPDP). Amide and thioether bonds, for example achieved with m-maleimidobenzoyl-N-hydroxysuccinimide ester, are generally more stable in vivo than disulphide bonds.

Alternatively, the agent may be produced as a fusion compound by recombinant DNA techniques whereby a length of DNA comprises respective regions encoding the two moieties of the agent of the invention either adjacent one another or separated by a region encoding a linker peptide which does not destroy the desired properties of the agent. Conceivably, the two portions of the agent may overlap wholly or partly.

The cytotoxic moiety may be a radiosensitizer. Radiosensitizers include fluoropyrimidines, thymidine analogues, hydroxyurea, gemcitabine, fludarabine, nicotinamide, halogenated pyrimidines, 3-aminobenzamide, 3-aminobenzodiamide, etanixadole, pimonidazole and misonidazole (see, for example, McGinn et al (1996) J. Natl. Cancer Inst. 88, 1193-11203; Shewach & Lawrence (1996) Invest. New Drugs 14, 257-263; Horsman (1995) Acta Oncol. 34, 571-587; Shenoy & Singh (1992) Clin. Invest. 10, 533-551; Mitchell et al (1989) Int. J. Radiat. Biol. 56, 827-836; Iliakis & Kurtzman (1989) Int. J. Radiat. Oncol. Biol. Phys. 16, 1235-1241; Brown (1989) Int. J. Radiat. Oncol. Biol. Phys. 16, 987-993; Brown (1985) Cancer 55, 2222-2228).

Also, delivery of genes into cells can radiosensitise them, for example delivery of the p53 gene or cyclin D (Lang et al (1998) J. Neurosurg. 89, 125-132; Coco Martin et al (1999) Cancer Res. 59, 1134-1140).

The further moiety may be one which becomes cytotoxic, or releases a cytotoxic moiety, upon irradiation. For example, the boron-10 isotope, when appropriately irradiated, releases a particles which are cytotoxic (for example, see U.S. Pat. No. 4,348,376 to Goldenberg; Primus et al (1996) Bioconjug. Chem. 7, 532-535).

Similarly, the cytotoxic moiety may be one which is useful in photodynamic therapy such as photofrin (see, for example, Dougherty et al (1998) J. Natl. Cancer Inst. 90, 889-905).

The further moiety may comprise a nucleic acid molecule which is directly or indirectly cytotoxic. For example, the nucleic acid molecule may be an antisense oligonucleotide which, upon localisation at the target site is able to enter cells and lead to their death. The oligonucleotide, therefore, may be one which prevents expression of an essential gene, or one which leads to a change in gene expression which causes apoptosis. Alternatively, the cytotoxic moiety is a nucleic acid molecule encoding a directly and/or indirectly cytotoxic polypeptide.

Examples of suitable oligonucleotides include those directed at bcl-2 (Ziegler et al (1997) J. Natl. Cancer Inst. 89, 1027-1036), and DNA polymerase α and topoisomerase IIα (Lee et al (1996) Anticancer Res. 16, 1805-1811.

Peptide nucleic acids may be useful in place of conventional nucleic acids (see Knudsen & Nielsen (1997) Anticancer Drugs 8, 113-118).

In a further embodiment, the binding moiety may be comprised in a delivery vehicle for delivering nucleic acid to the target. The delivery vehicle may be any suitable delivery vehicle. It may, for example, be a liposome containing nucleic acid, or it may be a virus or virus-like particle which is able to deliver nucleic acid. In these cases, the binding moiety is typically present on the surface of the delivery vehicle. For example, the binding moiety, such as a suitable antibody fragment, may be present in the outer surface of a liposome and the nucleic acid to be delivered may be present in the interior of the liposome. As another example, a viral vector, such as a retroviral or adenoviral vector, is engineered so that the binding moiety is attached to or located in the surface of the viral particle thus enabling the viral particle to be targeted to the desired site. Targeted delivery systems are also known such as the modified adenovirus system described in WO 94/10323 wherein, typically, the DNA is carried within the adenovirus, or adenovirus-like, particle. Michael et al (1995) Gene Therapy 2, 660-668 describes modification of adenovirus to add a cell-selective moiety into a fibre protein. Targeted retroviruses are also available for use in the invention; for example, sequences conferring specific binding affinities may be engineered into pre-existing viral env genes (see Miller & Vile (1995) Faseb J. 9, 190-199 for a review of this and other targeted vectors for gene therapy).

Immunoliposomes (antibody-directed liposomes) may be used in which the binding moiety is an antibody. For the preparation of immuno-liposomes MPB-PE (N-[4-(p-maleimidophenyl)-butyryl]-phosphatidylethanolamine) is synthesised according to the method of Martin & Papahadjopoulos (1982) J. Biol. Chem. 257, 286-288. MPB-PE is incorporated into the liposomal bilayers to allow a covalent coupling of the antibody, or fragment thereof, to the liposomal surface. The liposome is conveniently loaded with the DNA or other genetic construct for delivery to the target cells, for example, by forming the said liposomes in a solution of the DNA or other genetic construct, followed by sequential extrusion through polycarbonate membrane filters with 0.6 μm and 0.2 μm pore size under nitrogen pressures up to 0.8 MPa. After extrusion, entrapped DNA construct is separated from free DNA construct by ultracentrifugation at 80 000×g for 45 min. Freshly prepared MPB-PE-liposomes in deoxygenated buffer are mixed with freshly prepared antibody (or fragment thereof) and the coupling reactions are carried out in a nitrogen atmosphere at 4° C. under constant end over end rotation overnight. The immunoliposomes are separated from unconjugated antibodies by ultracentrifugation at 80 000×g for 45 min. Immunoliposomes may be injected intraperitoneally or directly into the tumour.

The nucleic acid delivered to the target site may be any suitable DNA which leads, directly or indirectly, to cytotoxicity. For example, the nucleic acid may encode a ribozyme which is cytotoxic to the cell, or it may encode an enzyme which is able to convert a substantially non-toxic prodrug into a cytotoxic drug (this latter system is sometime called GDEPT: Gene Directed Enzyme Prodrug Therapy).

Ribozymes which may be encoded in the nucleic acid to be delivered to the target are described in Cech and Herschlag “Site-specific cleavage of single stranded DNA” U.S. Pat. No. 5,180,818; Altman et al “Cleavage of targeted RNA by RNAse P” U.S. Pat. No. 5,168,053, Cantin et al “Ribozyme cleavage of HIV-1 RNA” U.S. Pat. No. 5,149,796; Cech et al “RNA ribozyme restriction endoribonucleases and methods”, U.S. Pat. No. 5,116,742; Been et al “RNA ribozyme polymerases, dephosphorylases, restriction endonucleases and methods”, U.S. Pat. No. 5,093,246; and Been et al “RNA ribozyme polymerases, dephosphorylases, restriction endoribonucleases and methods; cleaves single-stranded RNA at specific site by transesterification”, U.S. Pat. No. 4,987,071, all incorporated herein by reference. Suitable targets for ribozymes include transcription factors such as c-fos and c-myc, and bcl-2. Durai et al (1997) Anticancer Res. 17, 3307-3312 describes a hammerhead ribozyme against bcl-2.

EP 0 415 731 describes the GDEPT system. Similar considerations concerning the choice of enzyme and prodrug apply to the GDEPT system as to the ADEPT system described above.

The nucleic acid delivered to the target site may encode a directly cytotoxic polypeptide.

Alternatively, the further moiety may comprise a polypeptide or a polynucleotide encoding a polypeptide which is not either directly or indirectly cytotoxic but is of therapeutic benefit. Examples of such polypeptides include anti-proliferative or anti-inflammatory cytokines, and anti-proliferative, immunomodulatory or factors influencing blood clotting which may be of benefit in medicine, for example in the treatment of cancer.

The further moiety may usefully be an inhibitor of angiogenesis such as the peptides angiostatin or endostatin. The further moiety may also usefully be an enzyme which converts a precursor polypeptide to angiostatin or endostatin. Human matrix metallo-proteases such as macrophage elastase, gelatinase and stromolysin convert plasminogen to angiostatin (Cornelius et al (1998) J. Immunol. 161, 6845-6852). Plasminogen is a precursor of angiostatin.

In a preferred embodiment, the invention provides an agent further comprising a readily detectable moiety.

By a “readily detectable moiety” we include the meaning that the moiety is one which, when located at the target site following administration of the agent of the invention into a patient, may be detected, typically non-invasively from outside the body and the site of the target located. Thus, the agents of this embodiment of the invention are useful in imaging and diagnosis.

Typically, the readily detectable moiety is or comprises a radioactive atom which is useful in imaging. Suitable radioactive atoms include ^{99m}Tc and ¹²³I for scintigraphic studies. Other readily detectable moieties include, for example, spin labels for magnetic resonance imaging (MRI) such as ¹²³I again, ¹³¹I, ¹¹¹In, ¹⁹F, ¹³C, ¹⁵N, ¹⁷O, gadolinium, manganese or iron. Clearly, the agent of the invention must have sufficient of the appropriate atomic isotopes in order for the molecule to be readily detectable.

The radio- or other labels may be incorporated in the agent of the invention in known ways. For example, if the binding moiety is a polypeptide it may be biosynthesised or may be synthesised by chemical amino acid synthesis using suitable amino acid precursors involving, for example, fluorine-19 in place of hydrogen. Labels such as ^{99m}Tc, ¹²³I, ¹⁸⁶Rh, ¹⁸⁸Rh and ¹¹¹In can, for example, be attached via cysteine residues in the binding moiety. Yttrium-90 can be attached via a lysine residue. The IODOGEN method (Fraker et al (1978) Biochem. Biophys. Res. Comm. 80, 49-57) can be used to incorporate ¹²³I. Reference (“Monoclonal Antibodies in Immunoscintigraphy”, J-F Chatal, CRC Press, 1989) describes other methods in detail.

Preferably, the readily detectable moiety comprises a radioactive atom, such as, for example technetium-99m or iodine-123.

Alternatively, the readily detectable moiety may be selected from the group comprising: iodine-123; iodine-131; indium-111; fluorine-19; carbon-13; nitrogen-15; oxygen-17; gadolinium; manganese; iron.

In a preferred embodiment, the invention provides an agent further comprising a moiety capable of selectively binding to a directly or indirectly cytotoxic moiety.

In a further preferred embodiment of the invention the further moiety is able to bind selectively to a directly or indirectly cytotoxic moiety or to a readily detectable moiety. Thus, in this embodiment, the further moiety may be any moiety which binds to a further compound or component which is cytotoxic or readily detectable.

The further moiety may, therefore be an antibody which selectively binds to the further compound or component, or it may be some other binding moiety such as streptavidin or biotin or the like. The following examples illustrate the types of molecules that are included in the invention; other such molecules are readily apparent from the teachings herein.

A bispecific antibody wherein one binding site comprises the binding moiety (which selectively binds to the Ku protein defined herein) and the second binding site comprises a moiety which binds to, for example, an enzyme which is able to convert a substantially non-toxic prodrug to a cytotoxic drug.

Alternatively, the agent may comprise an antibody which selectively binds to the Ku protein defined herein, to which is bound biotin. Avidin or streptavidin which has been labelled with a readily detectable label may be used in conjunction with the biotin labelled antibody in a two-phase imaging system wherein the biotin labelled antibody is first localised to the target site in the patient, and then the labelled avidin or streptavidin is administered to the patient. Bispecific antibodies and biotin/streptavidin (avidin) systems are reviewed by Rosebrough (1996) Q J Nucl. Med. 40, 234-251.

In a preferred embodiment of the invention, the binding moiety and the further moiety are polypeptides which are fused.

Preferably, the invention provides an agent wherein the binding moiety comprises a peptide and/or a polypeptide.

Polypeptide binding moieties can be identified by means of a screen. A suitable method or screen for identifying peptides or other molecules which selectively bind a target protein or polypeptide may comprise contacting the target protein or polypeptide with a test peptide or other molecule under conditions where binding can occur, and then determining if the test molecule or peptide has bound the target protein or peptide. Methods of detecting binding between two moieties are well known in the art of biochemistry. Preferably, the known technique of phage display is used to identify peptides or other ligand molecules suitable for use as binding moieties. An alternative method includes the yeast two-hybrid system.

In one embodiment, the invention provides an agent wherein the binding moiety comprises a polypeptide sequence selected from the group comprising: SEQ ID NO:3; SEQ ID NO:4; SEQ ID NO:5. SEQ ID NO:6; SEQ ID NO:7; SEQ ID NO:8 SEQ ID NO:9; SEQ ID NO:10 and SEQ ID NO:11.

Preferably, the invention provides an agent wherein the binding moiety comprises an antibody or a fragment thereof.

By “antibody” we include not only whole immunoglobulin molecules but also fragments thereof such as Fab, F(ab′)2, Fv and other fragments thereof that retain the antigen-binding site. Similarly the term “antibody” includes genetically engineered derivatives of antibodies such as single chain Fv molecules (scFv) and single domain antibodies (dAbs). The term also includes antibody-like molecules which may be produced using phage-display techniques or other random selection techniques for molecules. The term also includes all classes of antibodies, including: IgG, IgA, IgM, IgD and IgE.

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

Antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dabs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we include molecules wherein the V_{H} and V_{L} partner domains are linked via a flexible oligopeptide.

The advantages of using antibody fragments, rather than whole antibodies, are several-fold. The smaller size of the fragments may lead to improved pharmacological properties, such as better penetration to the target site. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining site.

Although the antibody may be a polyclonal antibody, it is preferred if it is a monoclonal antibody. In some circumstances, particularly if the antibody is going to be administered repeatedly to a human patient, it is preferred if the monoclonal antibody is a human monoclonal antibody or a humanised monoclonal antibody.

Suitable monoclonal antibodies may be prepared by known techniques, for example those disclosed in “Monoclonal Antibodies; A manual of techniques”, H Zola (CRC Press, 1988) and in “Monoclonal Hybridoma Antibodies. Techniques and Application”, S G R Hurrell (CRC Press, 1982). Polyclonal antibodies may be produced which are polyspecific or monospecific. It is preferred that they are monospecific.

Chimeric antibodies are discussed by Neuberger et al (1998, 8^{th} International Biotechnology Symposium Part 2, 792-799).

Suitably prepared non-human antibodies can be “humanised” in known ways, for example by inserting the CDR regions of mouse antibodies into the framework of human antibodies.

The antibodies may be human antibodies in the sense that they have the amino acid sequence of human antibodies with specificity for the Ku protein defined herein but they may be prepared using methods known in the art that do not require immunisation of humans. For example, transgenic mice are available which contain, in essence, human immunoglobulin genes (see Vaughan et al (1998) Nature Biotechnol. 16, 535-539.

Preferably, the invention provides an agent wherein the antibody or fragment thereof is an scFv and/or a Fab. More preferably, the scFv comprises the polypeptide sequence of SEQ ID NO:11.

In a further embodiment, the invention provides the use of the agent of the invention in the manufacture of a medicament for the treatment of cancer. Methods of manufacturing a medicament using an active agent, such as the agent of the invention, are well known to persons skilled in the art of medicine and pharmacy.

Preferably, the invention provides a use wherein the cancer is a solid tumour and/or a carcinoma. More preferably, the invention provides a use wherein the cancer is selected from the group comprising: prostate carcinoma; breast carcinoma; colorectal carcinoma; pancreatic carcinoma; lung carcinoma; ovarian carcinoma; rhabdomyosarcoma; neuroblastoma; cervix epitheloid carcinoma; multiple myelomas; acute monocytic leukaemia; acute lymphoblastic leukaemia; glioblastoma.

In a second aspect, the invention provides an agent comprising a binding moiety capable of selectively binding to Ku protein as defined herein.

In a third aspect, the invention provides a pharmaceutical composition comprising a therapeutically effective amount of an agent according to the invention and a pharmaceutically-acceptable carrier.

By “therapeutically effective amount” we include an amount of the agent of the invention that is sufficient to induce and/or increase intracellular internalisation of the Ku protein and/or complex comprising the agent and Ku protein and thereby have a therapeutic effect. An effective amount could be determined in vivo by using the methods described in the Examples (for example, the methods used to determine internalisation of the INCA-X antibody and immunotoxin-directed cell cytotoxicity). A “therapeutic effect” is any effect that alleviates and/or prevents a condition associated with a disease, illness or condition and will vary depending on the condition to be treated. Appropriate tests for determining the therapeutic effect of an agent, composition or medicament of the invention will be known to those skilled in the relevant arts of medicine.

By “pharmaceutically acceptable” is included that the formulation is sterile and pyrogen free. Suitable pharmaceutical carriers are well known in the art of pharmacy. The carrier(s) must be “acceptable” in the sense of being compatible with the agent of the invention and not deleterious to the recipients thereof. Typically, the carriers will be water or saline which will be sterile and pyrogen free; however, other acceptable carriers may be used.

In a fourth aspect, the invention provides the use of an agent of the invention in the manufacture of a composition for the identification of cancer cells potentially susceptible to treatment using an agent as defined herein and/or a medicament as defined herein and/or a pharmaceutical composition as defined herein.

It will be understood by those skilled in the relevant arts of molecular and cellular biology that the agent of the invention could be used to identify cells, such as cancer cells, potentially susceptible to treatment using an agent as defined herein and/or a medicament as defined herein and/or a pharmaceutical composition as defined herein. For example, the agent of the invention could be used (in accordance with the methods of the invention and those described in the accompanying Examples) as a diagnostic reagent to detect the presence of the Ku protein on the surface of cells, such as cancer cells, in a test sample and monitor whether intracellular internalisation of such Ku protein is induced and/or increased upon binding of the agent. It would be clear to a skilled person that the identification of Ku protein on the surface of a cell and induction/increase of intracellular internalisation of the Ku protein may indicate that such a cell would be suitable for delivering a cytotoxic moiety using the agent of the invention and therefore susceptible to treatment using the agent and/or medicament and/or pharmaceutical composition of the invention.

In a fifth aspect, the invention provides a method for treating cancer in an individual comprising the step of administering to the individual an effective amount of an agent of the invention and/or a medicament of the invention and/or a pharmaceutical composition of the invention.

By “effective amount” we include an amount of the agent of the invention that is sufficient to prevent and/or reduce a condition associated with the particular cancer to be treated. For example, an effective amount may prevent and/or reduce the size and/or rate of growth and/or spread of a tumour. An effective amount may prevent and/or reduce the cellular differentiation of a cancer cell, thereby preventing and/or reducing metastasis. An effective amount could be determined in vivo by using the methods described in the Examples (for example, the methods used to determine internalisation of the INCA-X antibody and immunotoxin-directed cell cytotoxicity). Appropriate tests for determining the therapeutic effect and most appropriate route of administration of an agent, composition or medicament of the invention will be known to those skilled in the relevant arts of medicine

In a sixth aspect, the invention provides a method for identifying an individual having cancer cells potentially susceptible to treatment using an agent of the invention and/or a medicament of the invention and/or a pharmaceutical composition of the invention, the method comprising the steps of:
a) providing a sample comprising one or more cancer cell from the individual to be tested;
b) combining the sample with an agent according to the invention and/or a medicament according to the invention and/or a pharmaceutical composition according to the invention;
c) determining binding of the agent and/or medicament and/or pharmaceutical composition to Ku protein as defined herein localised on the surface of the one or more cancer cell, and subsequent intracellular internalisation of the Ku protein;
d) identifying an individual having cancer cells potentially susceptible to treatment in the event that the agent and/or medicament and/or pharmaceutical composition induces and/or promotes intracellular internalisation of Ku protein localised on the surface of the one or more cancer cell.

Preferably, the individual is a human, but may be any mammal such as a domesticated mammal (preferably of agricultural or commercial significance including a horse, pig, cow, sheep, dog and cat).

It will be understood by those skilled in the relevant arts of molecular and cellular biology and medicine that the agent of the invention could be used to identify an individual having cancer cells potentially susceptible to treatment using an agent as defined herein and/or a medicament as defined herein and/or a pharmaceutical composition as defined herein.

As discussed above, the agent of the invention could be used (in accordance with the methods of the invention and those described in the accompanying Examples) as a diagnostic reagent to detect the presence of the Ku protein on the surface of cells, such as cancer cells (either in a test sample obtained from the individual, or directly in the individual itself) and monitor whether intracellular internalisation of such Ku protein is induced and/or increased upon binding of the agent.

In a seventh aspect, the invention provides a method for identifying an agent capable of selectively binding to Ku protein as described herein localised on the surface of a cell and inducing and/or increasing intracellular internalisation of the Ku protein comprising the steps of:
a) providing a sample comprising Ku protein as described herein localised on the surface of one or more cell;
b) combining the sample with an agent to be tested;
c) determining whether the agent binds to Ku protein localised on the surface of the one or more cell, and whether Ku protein is subsequently internalised;
d) identifying an agent in the event that the agent is capable of selectively binding to Ku protein localised on the surface of a cell and inducing and/or increasing intracellular internalisation of the Ku protein.

Methods suitable for identifying an agent capable of selectively binding to Ku protein localised on the surface of a cell and inducing and/or increasing intracellular internalisation of the Ku protein are described in the accompanying Examples (for example, the methods used to determine internalisation of the INCA-X antibody and immunotoxin-directed cell cytotoxicity).

One or more agent to be tested may be obtained from a library of candidate molecules, such as a small molecule library and/or a chemical entity and/or an antibody library.

Preferably, the method of the seventh aspect of the invention further comprises the step of: (e) synthesising and/or isolating the agent identified in step (d).

Methods for synthesising an agent identified by the method of the seventh aspect of the invention will be well-known to those skilled in the art of biochemistry and chemistry.

Preferably, the method of the seventh aspect of the invention further comprises the step of formulating the agent identified in step (d) and/or synthesised and/or isolated in step (e) into a pharmaceutical composition.

Methods for formulating an agent into a pharmaceutical composition will be well-known to those skilled in the arts of medicine and pharmacy. Examples of such methods and formulations are given in the accompanying Examples.

In an eighth aspect, the invention provides a nucleic acid molecule encoding a agent according to the invention or a binding moiety thereof. By “nucleic acid molecule” we include DNA, cDNA and mRNA molecules, which may be single- or double-stranded.

In a ninth aspect, the invention provides an expression vector comprising a nucleic acid molecule according to the eighth aspect of the invention. By “expression vector” we mean one which is capable, in an appropriate host, of expressing a polypeptide encoded by the nucleic acid molecule.

Such vectors may be useful in expressing the encoded agent of the invention or binding moiety thereof in a host cell for production of useful quantities of the agents of the invention.

A variety of methods have been developed to operably link nucleic acid molecules, especially DNA, to vectors, for example, via complementary cohesive termini. For instance, complementary homopolymer tracts can be added to the DNA segment to be inserted into the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, e.g. generated by endonuclease restriction digestion, is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerising activities.

The combination of these activities therefore generates blunt-ended DNA segments. The blunt-ended segments are then incubated with a larger molar excess of linker molecules in the presence of an enzyme that is able to catalyse the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease site are commercially available from a number of sources including International Biotechnologies Inc., New Haven, Conn., USA.

A desirable way to modify the DNA encoding the polypeptide of the invention is to use PCR. This method may be used for introducing the DNA into a suitable vector, for example by engineering in suitable restriction sites, or it may be used to modify the DNA in other useful ways as is known in the art.

In this method the DNA to be enzymatically amplified is flanked by two specific primers which themselves become incorporated into the amplified DNA. The said specific primers may contain restriction endonuclease recognition sites which can be used for cloning into expression vectors using methods known in the art.

The DNA (or in the case of retroviral vectors, RNA) is then expressed in a suitable host to produce a polypeptide comprising the agent of the invention or binding moiety thereof. Thus, the DNA encoding the polypeptide may be used in accordance with known techniques, appropriately modified in view of the teachings contained herein, to construct an expression vector, which is then used to transform an appropriate host cell for the expression and production of the agent of the invention or binding moiety thereof. Such techniques include those disclosed in U.S. Pat. Nos. 4,440,859 issued 3 Apr. 1984 to Rutter et al, 4,530,901 issued 23 Jul. 1985 to Weissman, 4,582,800 issued 15 Apr. 1986 to Crowl, 4,677,063 issued 30 Jun. 1987 to Mark et al, 4,678,751 issued 7 Jul. 1987 to Goeddel, 4,704,362 issued 3 Nov. 1987 to Itakura et al, 4,710,463 issued 1 Dec. 1987 to Murray, 4,757,006 issued 12 Jul. 1988 to Toole, Jr. et al, 4,766,075 issued 23 Aug. 1988 to Goeddel et al and 4,810,648 issued 7 Mar. 1989 to Stalker, all of which are incorporated herein by reference.

The DNA (or in the case or retroviral vectors, RNA) encoding the polypeptide constituting the agent of the invention or binding moiety thereof may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the expression vector of the invention are then cultured for a sufficient time and under appropriate conditions known to those skilled in the art in view of the teachings disclosed herein to permit the expression of the polypeptide, which can then be recovered.

Many expression systems are known, including bacteria (for example, Escherichia coli and Bacillus subtilis), yeasts (for example Saccharomyces cerevisiae), filamentous fungi (for example Aspergillus), plant cells, animal cells and insect cells.

The vectors typically include a prokaryotic replicon, such as the ColE1 ori, for propagation in a prokaryote, even if the vector is to be used for expression in other, non-prokaryotic, cell types. The vectors can also include an appropriate promoter such as a prokaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical prokaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

A typical mammalian cell vector plasmid is pSVL available from Pharmacia, Piscataway, N.J., USA. This vector uses the SV40 late promoter to drive expression of cloned genes, the highest level of expression being found in T antigen-producing cells, such as COS-1 cells.

An example of an inducible mammalian expression vector is pMSG, also available from Pharmacia. This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, TRP1, LEU2 and URA3. Plasmids pRS413-416 are Yeast Centromere plasmids (Ycps).

Other vectors and expression systems are well known in the art for use with a variety of host cells.

In a tenth aspect, the invention provides a recombinant host cell comprising a nucleic acid molecule according to the eighth aspect of the invention.

Preferably, the recombinant host cell is a bacterial cell. Alternatively, the recombinant host cell is a mammalian cell.

The host cell can be either prokaryotic or eukaryotic. Bacterial cells are preferred prokaryotic host cells and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No. ATCC 31343). Preferred eukaryotic host cells include yeast, insect and mammalian cells, preferably vertebrate cells such as those from a mouse, rat, monkey or human fibroblastic and kidney cell lines. Yeast host cells include YPH499, YPH500 and YPH501 which are generally available from Stratagene Cloning Systems, La Jolla, Calif. 92037, USA. Preferred mammalian host cells include Chinese hamster ovary (CHO) cells available from the ATCC as CRL 1658 and 293 cells which are human embryonic kidney cells. Preferred insect cells are Sf9 cells which can be transfected with baculovirus expression vectors.

Transformation of appropriate cell hosts with a DNA construct of the present invention is accomplished by well known methods that typically depend on the type of vector used. With regard to transformation of prokaryotic host cells, see, for example, Cohen et al (1972) Proc. Natl. Acad. Sci. USA 69, 2110 and Sambrook et al (1989) Molecular Cloning, A Laboratory Manual, Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA.

Electroporation is also useful for transforming and/or transfecting cells and is well known in the art for transforming yeast cells, bacterial cells, insect cells and vertebrate cells.

For example, many bacterial species may be transformed by the methods described in Luchansky et al (1988) Mol. Microbiol. 2, 637-646 incorporated herein by reference. The greatest number of transformants is consistently recovered following electroporation of the DNA-cell mixture suspended in 2.5 PEB using 6250V per cm at 25 μFD.

Methods for transformation of yeast by electroporation are disclosed in Becker & Guarente (1990) Methods Enzymol. 194, 182.

Successfully transformed cells, i.e. cells that contain a DNA construct of the present invention, can be identified by well-known techniques. For example, cells resulting from the introduction of an expression construct of the present invention can be grown to produce the polypeptide of the invention. Cells can be harvested and lysed and their DNA content examined for the presence of the DNA using a method such as that described by Southern (1975) J. Mol. Biol. 98, 503 or Berent et al (1985) Biotech. 3, 208. Alternatively, the presence of the protein in the supernatant can be detected using antibodies as described below.

In addition to directly assaying for the presence of recombinant DNA, successful transformation can be confirmed by well known immunological methods when the recombinant DNA is capable of directing the expression of the protein. For example, cells successfully transformed with an expression vector produce proteins displaying appropriate antigenicity.

Samples of cells suspected of being transformed are harvested and assayed for the protein using suitable antibodies.

The host cell may be a host cell within a non-human animal body. Thus, transgenic non-human animals which express an agent according to the invention (or a binding moiety thereof) by virtue of the presence of the transgene are included. Preferably, the transgenic non-human animal is a rodent such as a mouse. Transgenic non-human animals can be made using methods well known in the art.

In an eleventh aspect, the invention provides a method of producing an agent according to the invention or a binding moiety thereof comprising: expressing a nucleic acid molecule according to the eighth aspect of the invention or an expression vector according to the ninth aspect of the invention or culturing a host cell according to the tenth aspect of the invention.

It will be appreciated that where the agent comprises distinct moieties, for example binding and/or cytotoxic domains, those moieties may be encoded by one or more separate nucleic acid molecules.

Methods for cultivating host cells and isolating recombinant proteins are well known in the art. It will be appreciated that, depending on the host cell, the agents of the invention (or binding moieties thereof) produced may differ. For example, certain host cells, such as yeast or bacterial cells, either do not have, or have different, post-translational modification systems which may result in the production of forms of agents of the invention (or binding moieties thereof) which may be post-translationally modified in a different way.

It is preferred that agents of the invention (or binding moieties thereof) are produced in a eukaryotic system, such as a mammalian cell.

According to a less preferred embodiment, the agents of the invention (or binding moieties thereof) can be produced in vitro using a commercially available in vitro translation system, such as rabbit reticulocyte lysate or wheatgerm lysate (available from Promega). Preferably, the translation system is rabbit reticulocyte lysate. Conveniently, the translation system may be coupled to a transcription system, such as the TNT transcription-translation system (Promega). This system has the advantage of producing suitable mRNA transcript from an encoding DNA polynucleotide in the same reaction as the translation.

Preferably, the production method of this aspect of the invention comprises a further step of isolating the agents of the invention (or binding moieties thereof) produced from the host cell or from the in vitro translation mix. Preferably, the isolation employs an antibody which selectively binds the expressed polypeptide of the invention.

As discussed above, in a preferred embodiment of the agent of the invention the binding moiety comprises an antibody or antigen-binding fragment thereof. Antibodies can be raised in an animal by immunising with an appropriate peptide. Appropriate peptides include the proteins listed in Table 1 and fragments thereof. Alternatively, with today's technology, it is possible to make antibodies as defined herein without the need to use animals. Such techniques include, for example, antibody phage display technology as is well known in the art. Appropriate peptides, as described herein, may be used to select antibodies produced in this way.

It will be appreciated that, with advances in antibody technology, it may not be necessary to immunise an animal in order to produce an antibody. Synthetic systems, such as phage display libraries, may be used. The use of such systems is included in the methods of the invention and the products of such systems are “antibodies” for the purposes of the invention.

It will be appreciated that such antibodies which recognise one of the proteins listed in Table 1 and variants or fragments thereof are useful research reagents and therapeutic agents, particularly when prepared as a agent of the invention as described above. Suitably, the antibodies of the invention are detectably labelled, for example they may be labelled in such a way that they may be directly or indirectly detected. Conveniently, the antibodies are labelled with a radioactive moiety or a coloured moiety or a fluorescent moiety, or they may be linked to an enzyme. Typically, the enzyme is one which can convert a non-coloured (or non-fluorescent) substrate to a coloured (or fluorescent) product. The antibody may be labelled by biotin (or streptavidin) and then detected indirectly using streptavidin (or biotin) which has been labelled with a radioactive moiety or a coloured moiety or a fluorescent moiety, or the like or they may be linked to any enzyme of the type described above.

The invention also provides a kit of parts (or a therapeutic system) comprising an agent of the invention wherein the further moiety is able to bind selectively to a directly or indirectly cytotoxic moiety or to a readily detectable moiety, and any one of a directly or indirectly cytotoxic or a readily detectable moiety to which the further moiety of the agent is able to bind.

The invention also provides a kit of parts (or a therapeutic system) comprising an agent of the invention wherein the further moiety is able to bind selectively to a directly or indirectly cytotoxic moiety or to a readily detectable moiety, and any one of a directly or indirectly cytotoxic or a readily detectable moiety to which the further moiety of the agent is able to bind.

Preferred, non-limiting examples which embody certain aspects of the invention will now be described, with reference to the following figures:

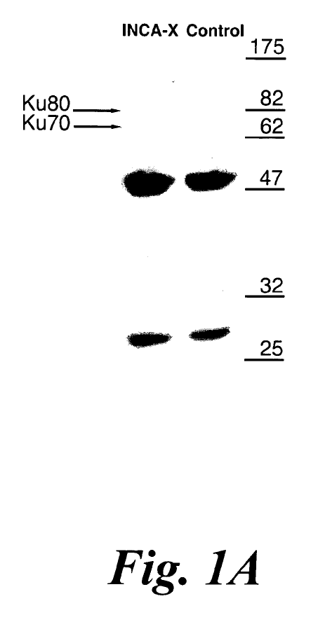

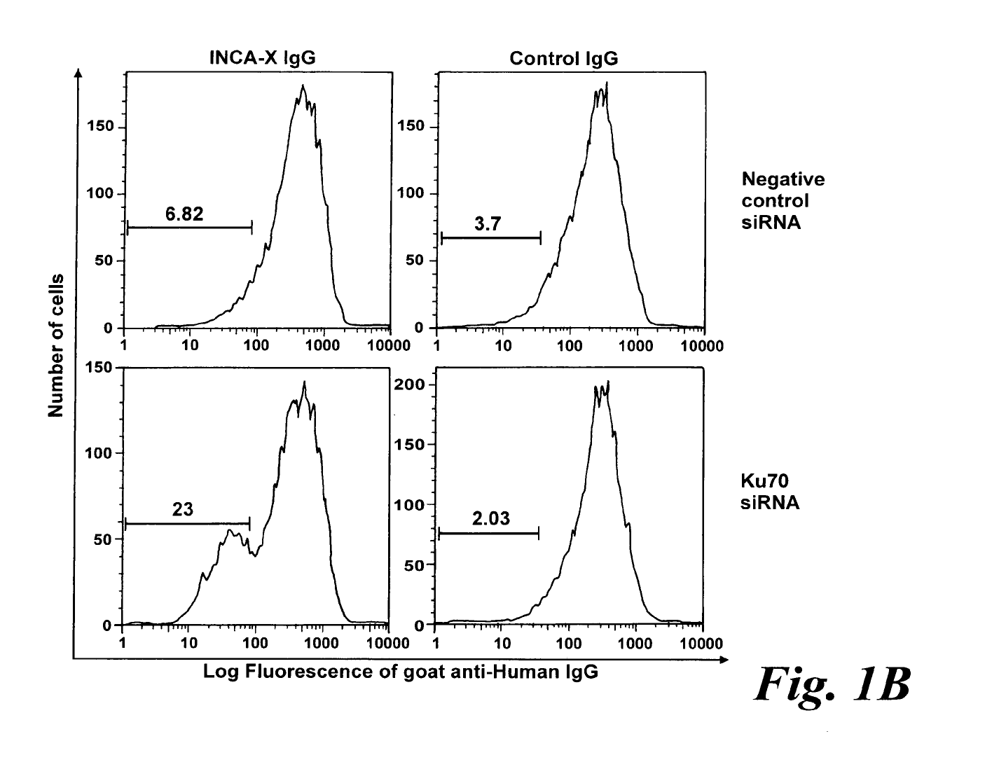

FIG. 1: The Target Antigen of the INCA-X Antibody is Ku70/80.

A. Solubilised membrane fractions of the human pancreatic adenocarcinoma cell line HPAC were immunoprecipitated by 20 μg of INCA-X or a control IgG antibody, as described in M & M. Samples were run on 4-12% SDS-PAGE and unique protein bands were cut from the gel and subjected to trypsin digestion and subsequent MALDI-TOF analysis. B. Adherently growing HPAC cells were transfected with 50 nM siRNA targeting either the Ku70 mRNA transcript or a non-existing transcript, i.e. negative control siRNA. 24 hours after transfection, cells were harvested and the binding antibodies were analyzed by flow cytometry. Bars show the percent of gated cell population.

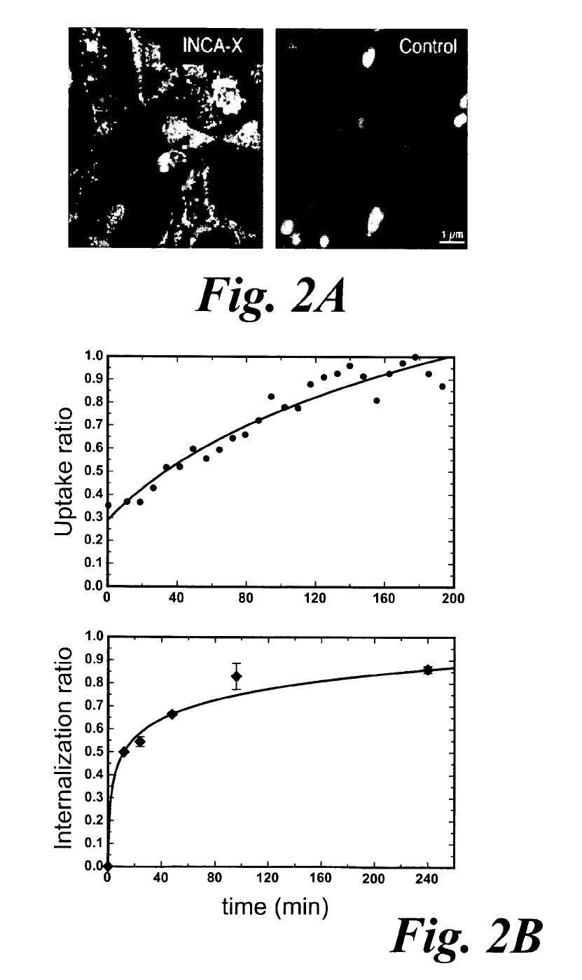

FIG. 2: Antibody-Induced Receptor-Mediated Endocytosis in Pancreatic Adenocarcinoma Cells.

A. PL45 cells were incubated with either (left) INCA-X IgG or (right) an irrelevant IgG antibody mixture. Internalization was determined by indirect staining with biotin-conjugated anti-human IgG antibody followed by Alexa-488 conjugated streptavidin, shown in green. Nuclei were stained with propidium iodide (red). Results were analyzed by confocal microscopy. B. INCA-X IgG was coupled to ¹²⁵I and added to HPAC cells in a rotating cell dish system. The binding (upper panel) of INCA-X to cells was monitored by recording the antibody signal in the area containing growing cells, compared to an empty reference area. In the internalization experiment (lower panel), the internalized ratio was determined by dividing the internalized activity with the total cell-associated activity (membrane-bound and internalized).

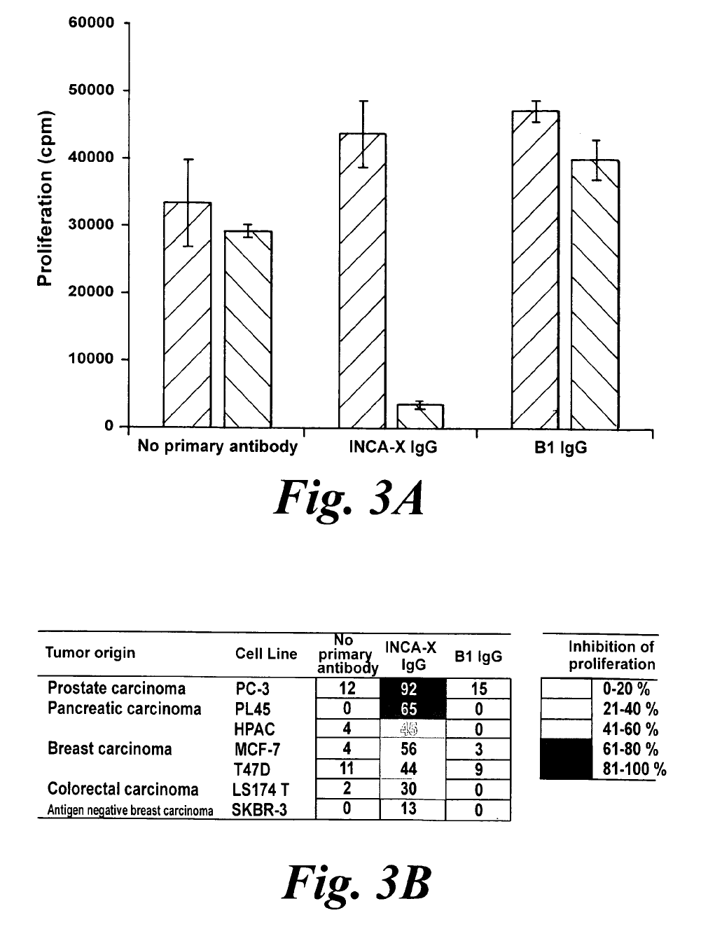

FIG. 3: Cell Growth Inhibition by an INCA-X Immunotoxin Conjugate.

A. Adherently growing PC-3 prostate carcinoma cells were incubated with 10 μg/ml saporin-conjugated anti-human antibody (Hum-ZAP, black lined columns) followed by the addition of INCA-X or B1, a non-binding control IgG1 antibody, at 10 nM final concentration. The ability of INCA-X (or control IgG1) to kill the carcinoma cells was assessed after 72 hours and compared to wells treated with primary antibody but no Hum-ZAP (gray lined columns). Bars ±SD. B. The indirect cytotoxic effect of INCA-X was determined, similarly as above, on various antigen-positive and negative cell lines. The inhibition of proliferation is given as the percentage of proliferation between wells with or without the addition of Hum-ZAP.

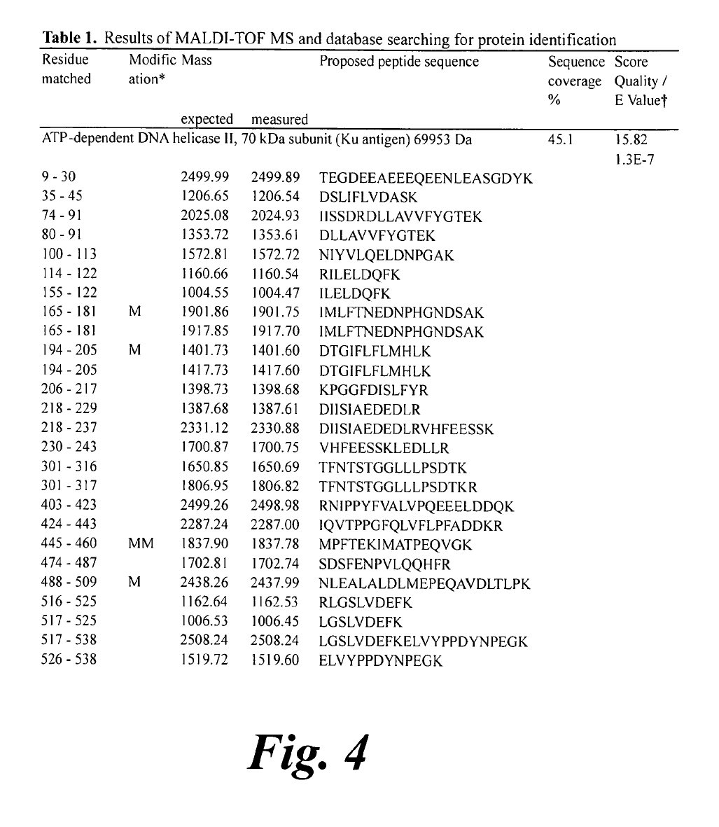

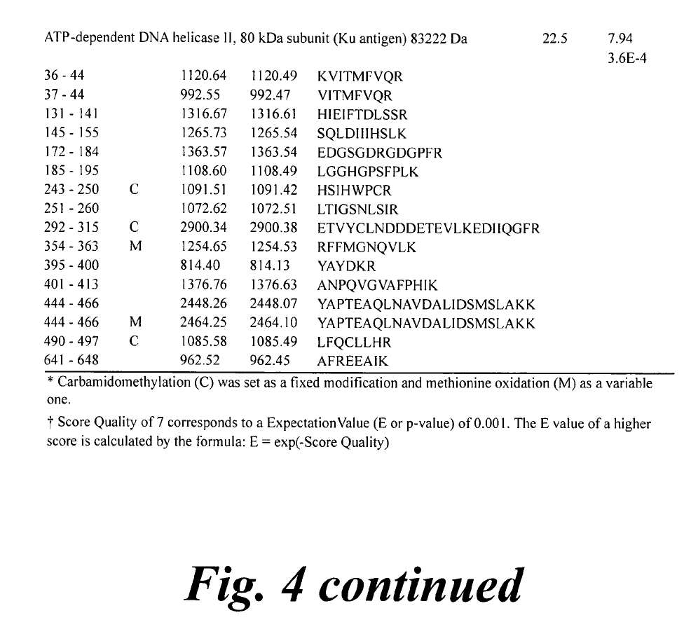

FIG. 4—Table 1: results of MALDI-TOF MS and database searching for protein identification.

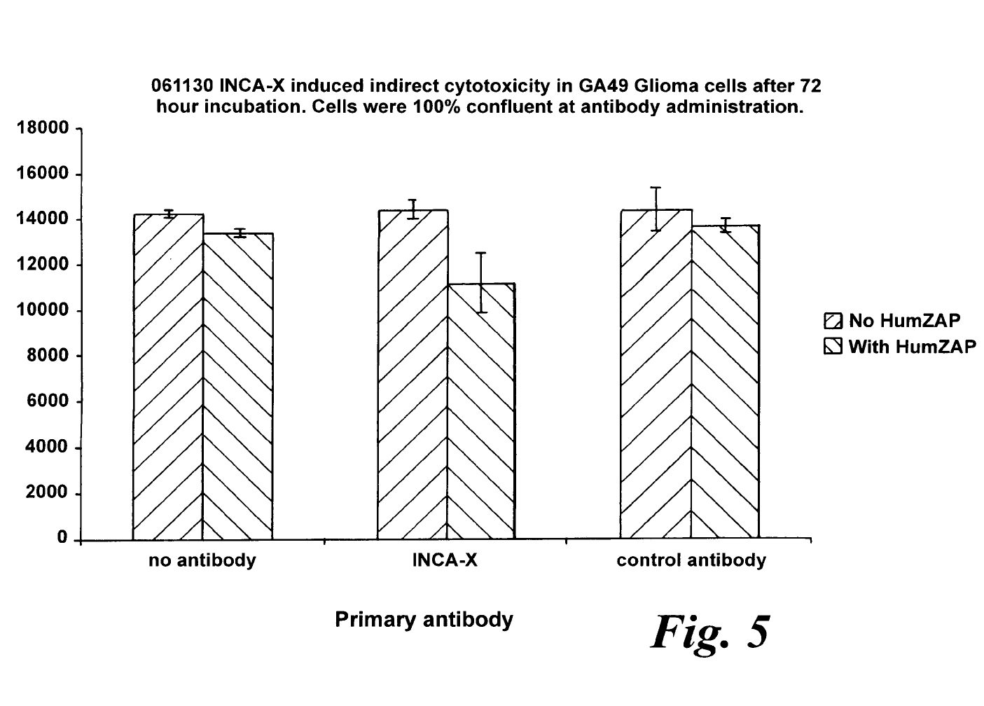

FIG. 5: INCA-X induced indirect cytotoxicity in GA49 Glioma cells after 72 hour incubation. Cells were 100% confluent at antibody administration.

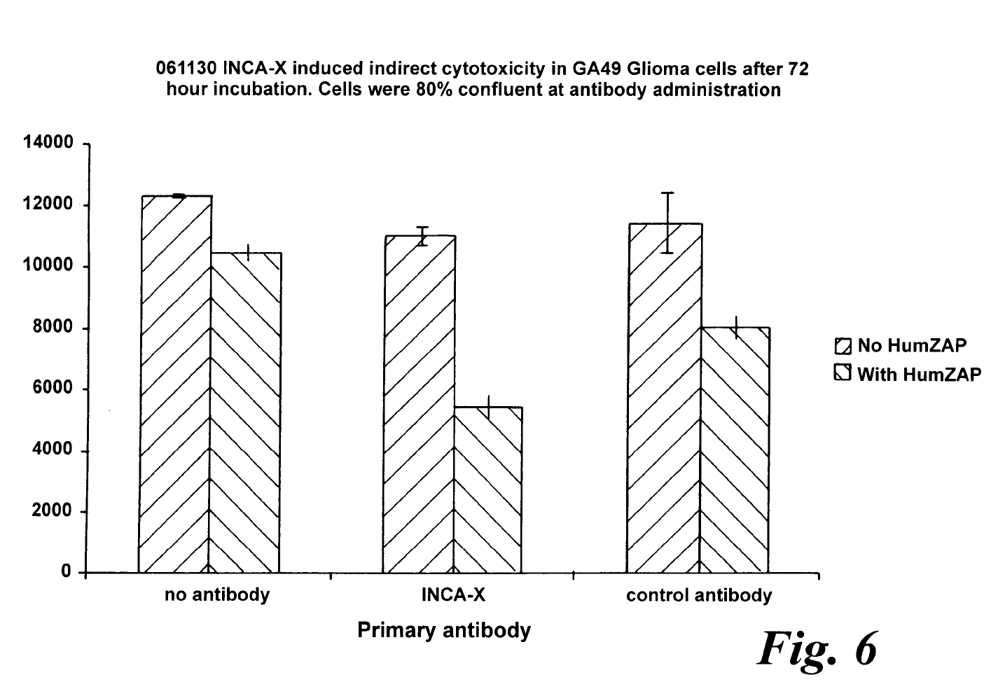

FIG. 6: INCA-X induced indirect cytotoxicity in GA49 Glioma cells after 72 hour incubation. Cells were 80% confluent at antibody administration.

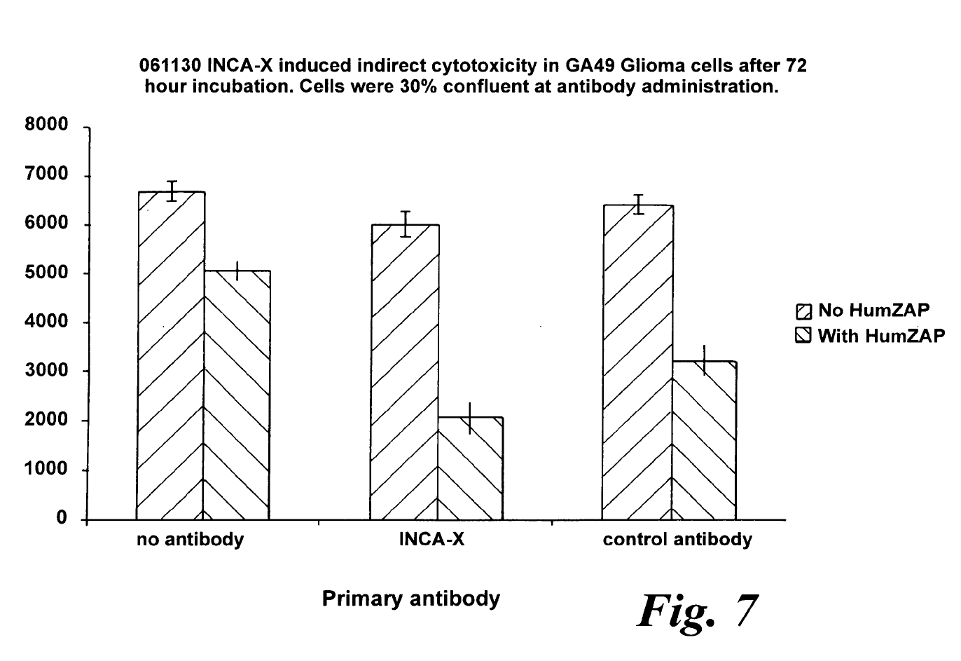

FIG. 7: INCA-X induced indirect cytotoxicity in GA49 Glioma cells after 72 hour incubation. Cells were 30% confluent at antibody administration.

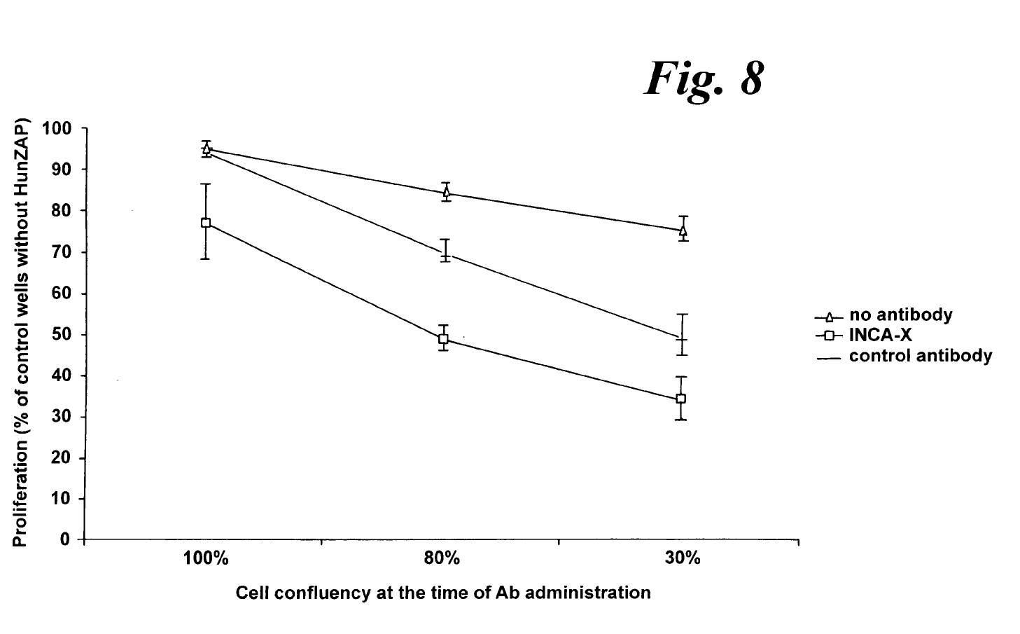

FIG. 8: INCA-X induced indirect cytotoxicity in GA49 Glioma cells with varying cell confluency.

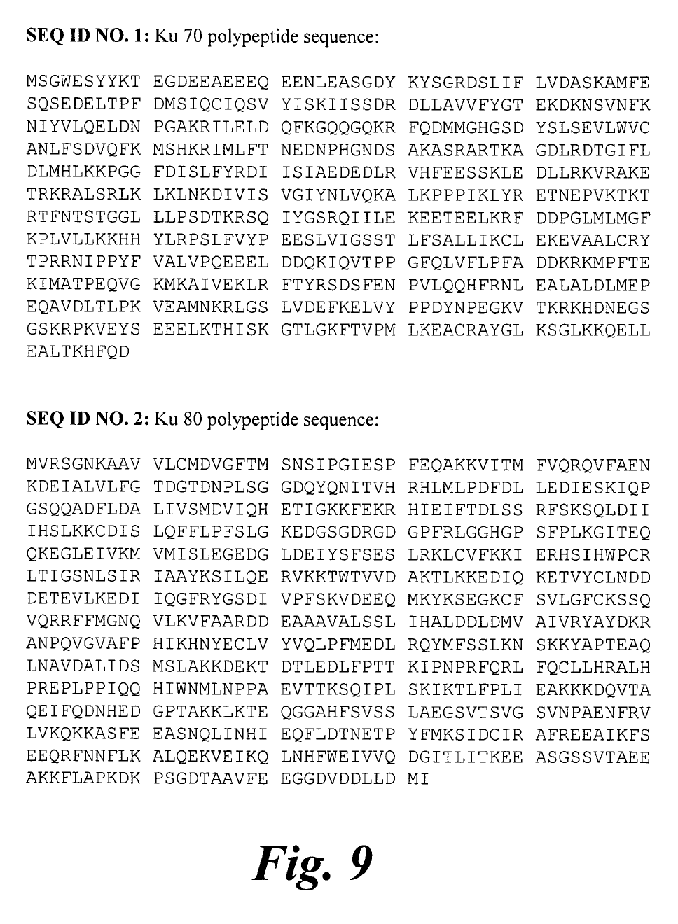

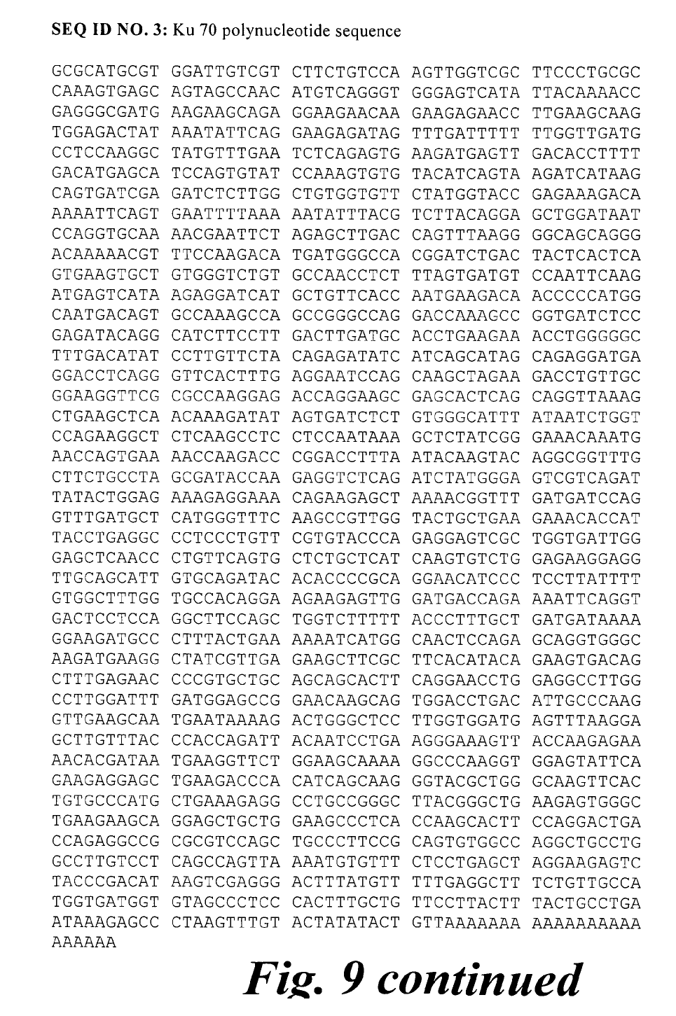

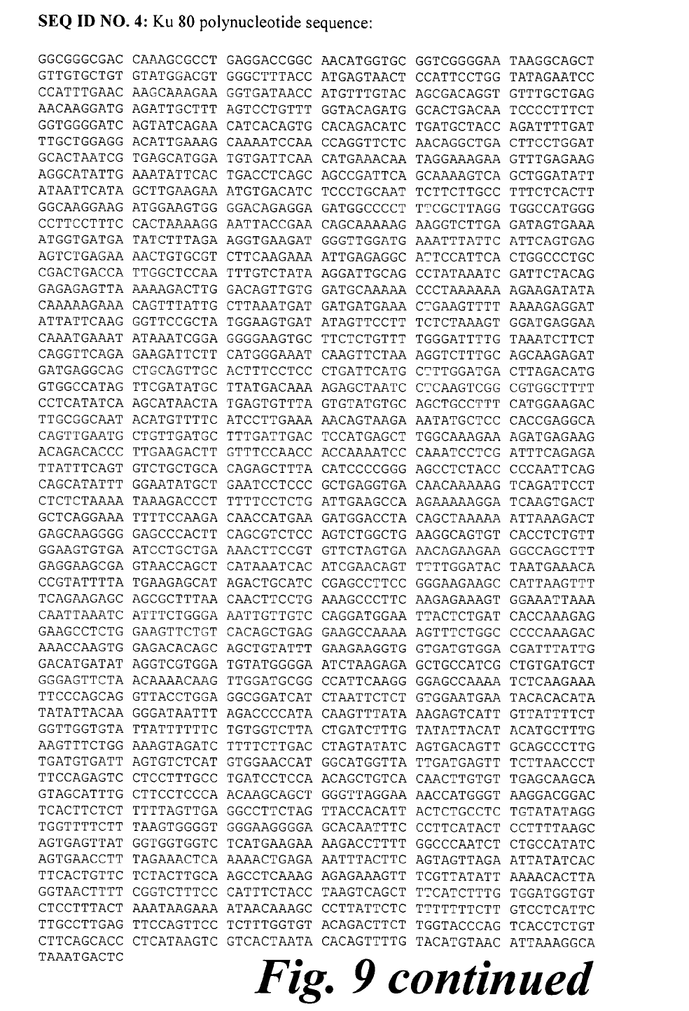

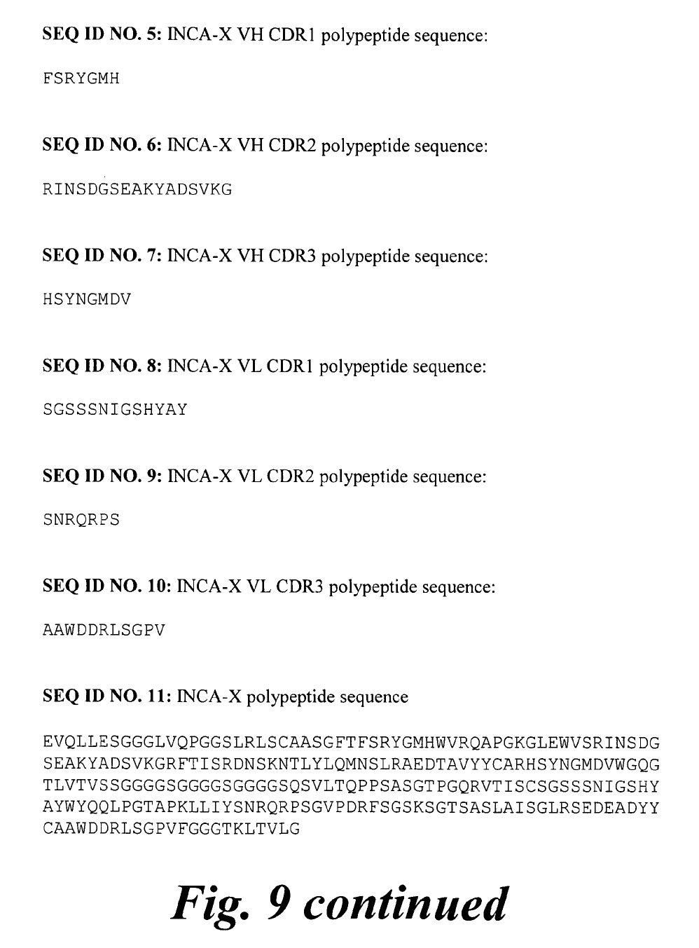

FIG. 9: Polynucleotide and polypeptide sequences

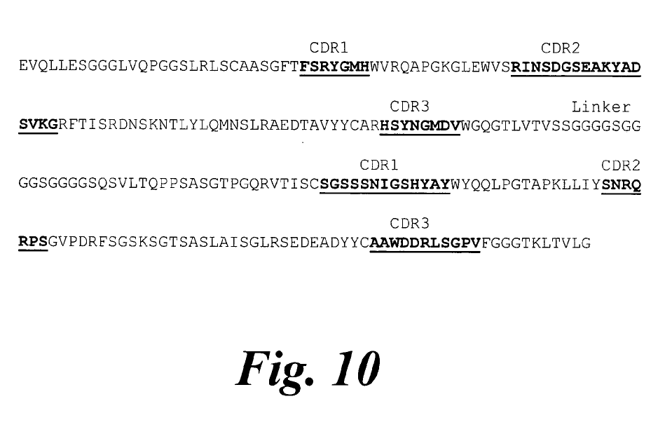

FIG. 10: INCA-X polypeptide sequence with CDR 1-3 (underlined) in VH and VL marked (according to Kabat E A et al. 1991, In “Sequences of Proteins of Immunological Interest” Fifth Edition, NIH Publication No. 91-3242, pp xv-xvii).

### EXAMPLE 1

### Experimental Data

### Material and Methods

### Cell Lines

The human pancreatic adenocarcinoma cell lines HPAC (CRL-2558) and PL45 (CRL-2119), the prostate carcinoma PC-3 (CRL-1435), the breast carcinomas SK-BR-3 (HTB-30), T-47D (HTB-133), MCF-7 (HTB-22), and the colorectal carcinoma LS 174T (CL-188) was obtained from ATCC (Manassas, Va.) and cultured according to the recommendations of the distributor.

### Isolation of a Human Antibody Against Ku 70/80

The isolation of internalizing human antibodies from the naïve phage antibody library n-CoDeR® (Söderlind et al., 2000) was performed using a technology recently described (Fransson, 2004). Briefly, the single chain antibody (scFv) library, displayed on phage, was first subtracted by incubation with the T cell leukemia cell line MOLT-4. Selections were performed on the pancreatic adenocarcinoma cell line PL45, during 1 h incubation at 37° C., allowing internalization of the phage scFv-receptor complexes. Internalized phages were retrieved by lysing surface-stripped cells with 100 mM triethylamine. The selection process generated several internalizing scFv, one which was denoted INCA-X, was reformatted to a fully human IgG1 antibody (Norderhaug et al., 1997).

### Membrane Fractionation and Immunoprecipitation

The identity of the INCA-X antigen was determined by immunoprecipitation of purified HPAC membrane fractions. Cells were washed twice in PBS and the cell pellet was resuspended in 5 ml Hypotonic Buffer (5 mM Tris-HCl, pH 7.5, 5 mM EDTA, and Complete EDTA-free Protease inhibitor cocktail (Roche, Mannheim, Germany)). The swelled cells were homogenized in a Dounce homogenizer on ice and subjected to a series of differential centrifugation steps. Nuclei and unbroken cells were pelleted by centrifugation at 1,000×g for 5 min. The supernatant was centrifuged at 10,000×g for 12 min, thereby pelleting intracellular organelles. The supernatant from the 10,000×g centrifugation was centrifuged at 100,000×g to generate a microsomal pellet. The membrane proteins were solubilised over night at 4° C. in 0.5% (v/v) NP40 Isotonic Buffer (25 mM Tris-HCl, pH 7.5, 5 mM EDTA, 150 mM NaCl, and protease inhibitor cocktail) followed by 100,000×g centrifugation where after the supernatant, containing the solubilised membrane proteins were stored at −20° C.

The antigen recognized by the INCA-X antibody was immunoprecipitated, as described earlier (Fransson et al, 2006). Briefly, the membrane fraction was pre-cleared with Protein A Sepharose 4 Fast Flow (Amersham Biosciences, Uppsala, Sweden) for 2 h on rotation and immunoprecipitated over night by 20 μg INCA-X antibody, or a human IgG control. Protein A Sepharose, pre-blocked in 1% w/v non-fat skimmed milk in Isotonic Buffer with 0.1% (v/v) NP40, was added and incubated for 1 h, where after the immuno-complexes were washed extensively in Isotonic Buffer with 0.1% NP40, boiled for 5 min, and separated in a 4-12% SDS-PAGE. After staining, protein bands of interest were excised from the SDS-PAGE and subjected to tryptic digestion, as described (Fransson et al., 2006). Peptide masses were determined using a M@LDI LRHT (Waters, Manchester, UK) mass spectrometer. Database searching was performed against the comprehensive, non-redundant IPI human database using the Piums software (Samuelsson et al., 2004).

### Functional Identification of Ku Antigen by RNA Interference

In order to confirm the MALDI-TOF results for the INCA-X antibody specificity, a validated small interfering RNA (siRNA) oligonucleotide towards the Ku (p70) transcript was purchased from Ambion (Austin, Tex.). Transfections of Ku70 siRNA into HPAC cells were performed using Lipofectamine 2000 (Invitrogen, Paisley, UK) according to the distributor's recommendations. 24 h post-transfection, binding of INCA-X or a control human IgG antibody to the HPAC cells was assessed by flow cytometry. The human IgGs were detected with PE-labeled Goat anti-Human IgG (Caltag Laboratories, Burlingame, Calif.) and analyzed in a FACScan instrument (Beckton Dickinson, Franklin Lakes, N.J.).

### Internalization of INCA-X

Internalization of the INCA-X antibody was verified by confocal microscopy, as described (Fransson et al., 2004). Briefly, INCA-X IgG was incubated with PL45 cells over night and internalized antibody was detected by incubating saporin-treated cells with biotin-conjugated goat anti-human IgG (Jackson ImmunoResearch Laboratories, West Grove, Pa.) and Alexa 488-conjugated streptavidin (Invitrogen). Cell nuclei were stained with propidium iodide and the internalization was analyzed using a BioRad Confocal Microscope (Hercules, Calif.).

Uptake, internalization and retention of the INCA-X antibody was performed on a LigandTracer™ prototype (Ridgeview Instruments AB, Uppsala, Sweden) (Björke and Andersson, 2006). The instrument measures radioligand-cell interactions by use of a rotating radioimmunoassay technology (RIA). INCA-X, radio labeled using the Chloramine-T method, as described by Sundberg et al (2003), were added in 1 ml growth medium to the cell dish and measurements were performed during indicated time-points. The detector was set to record five sample points in the area containing growing cells, and five points in the empty reference area. All measurements were carried out in a humidified incubator at 37° C. and 5% CO₂. In the internalization kinetic studies, the membrane bound activity was measured after stripping the cells with 0.1 M Glycine-HCl (pH 2.5) and the internalized activity was measured after lysing the cells with 1 M NaOH.

### Immunotoxin-Directed Cell Cytotoxicity.

To evaluate the potential role of INCA-X in immunotherapy, indirect immunotoxin experiments were carried out on several human tumor cell lines. Cells were plated at 10,000 per well (100,000/ml) in 96-well plates and incubated overnight at 37° C. in 5% CO₂. The cells were treated with 200 ng of INCA-X/well primary antibody. The antibodies used were the fully human IgG1 formats of INCA-X and a control antibody B1 (a human antibody reactive to human MHC class II) (Fransson et al., 2006). Half of the test wells were subsequently treated with 100 ng/well of saporin-conjugated anti-human antibody (Hum-ZAP, Advanced Targeting Systems, San Diego, Calif.). The cytotoxicity of the immunotoxin conjugate was assessed by a [³H]thymidine incorporation assay. Cells were pulsed after 56 hours by the addition of 0.5 μCi/well of [³H]thymidine (Amersham Biosciences) and incubated for 16 h. The incorporation of [³H]thymidine was determined by reading in a 1450 Micro Beta Liquid Scintillation Counter (Pharmacia, Uppsala, Sweden).

### Indirect Cytotoxicity of INCA-X Against Human Glioma GA49.

To investigate if INCA-X binding to GA49 cells (human primary glioma tumour cells) could be used to induce indirect cytotoxicity by a secondary saproin-conjugated antibody (HumZAP).

Materials:
HumZAP, goat anti-Human IgG (saporin conjugated), 2.9 mg/ml (Advanced Targeting Systems, San Diego, Calif.)
INCA-X, 2.5 mg/ml
Control antibody, 2.3 mg/ml (human anti-ICAM1 IgG₁ antibody).
GA49, human primary glioma tumour cells from Bengt Widegren, Dept of Immunology, Lund University, Lund, Sweden
Glioma Cell Medium: Iscove's Modified Eagles Medium (IMDM) (pour of 115 ml), 20% FCS (add 100 ml), MEM non essential amino acids; 100× (add 5 ml), Sodium Pyruvate (100 mM, add 5 ml), beta-mercaptoethanol (add 7 μl of stock solution) to 10 ml PBS, sterile filter through 0.2 μm. Add 5 ml to medium.

Procedure.
1. 24 hours prior to antibody administration, count cells and seed into 96 well plates. Seeding cells at varying dilutions of cells will obtain differing cell confluency. (Seed approx 10,000 cells/well/1001 medium).
2. At the time of experiment, dilute HumZAP in Glioma Cell Medium to 100 ng/well (10 μl per well of a 10 μg/ml solution).
3. Add diluted HumZAP to wells.
4. Dilute INCA-X and control antibody human (anti-ICAM1 IgG₁ antibody) to 20 μg/ml and add 10 μl per well (giving a final concentration of 2 μg/ml). Include control wells with medium only and HumZAP with no primary antibody. Set up duplicate samples.
5. Incubate for 3 days.
6. Add 100 μl 3H-Thymidine at a final concentration of 0.5 μCi/well. Pulse cells for 8 hours and put in freezer until harvesting.
7. Harvest cells in a beta-scintillation chamber.

### Results and Discussion

Cell surface receptors are key targets for antibody-based therapy of cancer. Upon antibody binding, the targeted membrane-associated antigens might relay signalling into the cells e.g. initiation of apoptosis (Shan et al., 1998; Fransson et al., 2006) or be internalized by receptor-mediated endocytosis (Liu et al., 2004; Fransson et al., 2004). Internalization of the receptor after antibody engagement opens a window for a wide range of therapeutic interventions, such as antibody-drug conjugates (Wu, 2005) or antibody mediated gene therapy (Zhang et al., 2002).

To investigate the possibility to generate internalizing human antibodies against unknown membrane bound antigens we applied our recently described approach, using the human pancreatic adenocarcinoma cell line PL45 (Fransson et al, 2004). In particular, one human antibody, INCA-X, which displayed a very rapid and robust internalization was characterized further. In order to define its antigen specificity, INCA-X was used to precipitate solubilised plasma membrane fractions of HPAC pancreatic adenocarcinoma cells. Immunoprecipitation, using the fully human IgG1 INCA-X antibody, resulted in two specific bands of 70 and 80 kDa size (FIG. 1A). The bands were cut and subjected to in-gel trypsination and analyzed by MALDI-TOF. The peptide mass fingerprints of the bands were identified as the two subunits of the ATP-dependent DNA helicase II protein, making up the Ku heterodimer (Ku70/Ku80) (FIG. 4/Table 1). The specificity of the INCA-X targeted antigen was confirmed by Western blots of precipitates of other Ku-specific antibodies generated by Monferran et al. (2004) (data not shown). Further confirmation studies were performed by transfecting HPAC cells with siRNA, targeting the Ku70 mRNA transcript, followed by assessment of the INCA-X antibody binding to transfected cells, using flow cytometry (FIG. 1B). In HPAC cells where the expression of Ku70 had been silenced by siRNA, two cell populations were identified 24 hours after transfection, i.e. INCA-X^{high} and INCA-X^{low}, of which the INCA-X^{low} population made up 23%. In wells treated with a negative control siRNA, or when an unspecific control antibody was used, no effect on the antigen expression could be detected (FIG. 1B). Thus, the INCA-X^{low} population represents the successfully siRNA silenced cells, displaying a substantial decreased amount of the membrane-associated Ku70 protein.

Ku70/80 has not previously been shown to mediate internalization in tumor cells, although Martinez et al. (Martinez, 2005) recently reported that Ku70 was functionally involved in the cellular uptake of the intracellular bacterial pathogen Rickettsia conorii. We therefore performed confocal microscopy clearly demonstrating that the INCA-X antibody internalized in pancreatic carcinoma cell lines (FIG. 2A). In order to determine the rate and extent of this receptor mediated endocytosis, the antibody was radiolabeled, using the Chloramine-T method (Sundberg et al., 2003) and added to a rotating cell dish system (Björke et al., 2006). This system allows for the measurement of binding to the cell membrane (uptake), internalization and retention kinetics. The uptake of [¹²⁵I]INCA-X was rapid, reaching a saturation plateau after 2 hours (FIG. 2B). Furthermore, 50% internalization of the [¹²⁵I]INCA-X was achieved already at the first time-point analyzed (12 mm) and after 100 min, approximately 90% of the antibody was located inside the cell (FIG. 2B). Finally, the half-life of INCA-X retention was determined to approximately 5 hours (data not shown). The high intracellular accumulation of INCA-X compares favorably to previously described internalizing antibody-antigen pairs. As a comparison, only 30% of the ¹²⁵I-labeled mAb 14C5 was internalized into lung and colon carcinoma cells during a 2 h incubation (Bunzenich et al., 2005). Furthermore, INCA-X has similar rate and degree of cellular uptake as the highly internalizing ⁶⁴Cu-DOTA-cBR96 antibody in the LS174T colon cancer cell line (Bryan et al., 2005).

The expression of the Ku heterodimer on human cell lines, as well as primary normal and tumor cells, has been recently reviewed by Muller et al. (2005). Ku70/80 is expressed in the nucleus of all cells, but its plasma membrane localization is restricted to tumor cell lines of different lineages, where it also might be upregulated under hypoxia or upon CD40L stimulation, whereas the only normal human primary cells expressing surface-associated Ku70/80 are macrophages and HUVECs (Muller, 2005). Hence, Ku70/80 has the potential to act as an optimal molecular target for immunotherapy. In order to corroborate the results from the internalization study and to assess its potential use as entry port for cytotoxic agents, the immunotoxin-mediated cell cytotoxicity of INCA-X was determined. Carcinoma cell lines, with varying surface expression of Ku70/80 as determined by flow cytometry (data not shown), were treated with 10 nM of INCA-X antibody or the isotype control IgG B1 antibody. After addition of the primary antibody, the cells were incubated with a saporin toxin-conjugated anti-human IgG antibody (Hum-ZAP). Upon internalization of the toxin complex, the saporin breaks away from the targeting agent and inactivates the ribosomes (Foehr et al., 2006). FIG. 3 demonstrates that INCA-X effectively kill Ku70/80-positive carcinoma cell lines, whereas the control antibody showed no cytotoxicity, which was comparable to wells treated with primary antibody alone. The inhibition of proliferation, induced by INCA-X/Hum-ZAP, ranged from very strong (92% on the prostate carcinoma PC-3) to intermediate (30% on the colorectal carcinoma LS174 T), whereas no effect was seen on the Ku70/80 antigen-negative, breast carcinoma cell line SK-BR-3.

FIGS. 5-8 show the effect of INCA-X and a control antibody (human anti-ICAM1 IgG₁ antibody) upon secondary saporin-conjugated antibody administration. After 72 hours, INCA-X showed a marked cytotoxic effect on the glioma cells GA49. The control antibody (human anti-ICAM1 IgG1 antibody) also induced some cytotoxicity, but whether the control antibody binds to the cell surface of these cells has not been evaluated. Cell confluency is an important factor and the effect of HumZAP was greater when cells with low confluency was subjected to cytotoxicity, although cells in control wells also suffered from some non-specific HumZAP uptake.

These data provide proof-of-concept for INCA-X, as a potential therapeutic immunoconjugate. In contrast to saporin, which due to its size and immunogenicity is not optimal in a clinical setting, the antibody-toxin molecule should have a linker that is cleaved inside the cell and a toxin that is potent, such as Doxorubicin, conjugated by the acid-labile linker cis-aconitic anhydride (Yang et al., 1988).

In conclusion, we report on a novel feature of the Ku70/80 heterodimer defined by the functional and extensive internalization into several human tumor cell lines, initiated by the human antibody INCA-X. Furthermore, INCA-X also induced extensive cell death when carrying a toxin as payload.

### EXAMPLE 2

### Preferred Pharmaceutical Formulations and Modes and Doses of Administration

The agents, medicaments and pharmaceutical compositions of the present invention may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

An alternative method of delivery of the agents, medicaments and pharmaceutical compositions of the invention is the ReGel injectable system that is thermo-sensitive. Below body temperature, ReGel is an injectable liquid while at body temperature it immediately forms a gel reservoir that slowly erodes and dissolves into known, safe, biodegradable polymers. The active substance is delivered over time as the biopolymers dissolve.

The agents, medicaments and pharmaceutical compositions of the invention can also be delivered orally. The process employs a natural process for oral uptake of vitamin B₁₂ in the body to co-deliver proteins and peptides. By riding the vitamin B₁₂ uptake system, the nucleic acids, molecules and pharmaceutical formulations of the invention can move through the intestinal wall. Complexes are synthesised between vitamin B₁₂ analogues and the drug that retain both significant affinity for intrinsic factor (IF) in the vitamin B₁₂ portion of the complex and significant bioactivity of the active substance of the complex.

In human therapy, the agents, medicaments and pharmaceutical compositions of the invention can be administered alone but will generally be administered in admixture with a suitable pharmaceutical excipient, diluent or carrier selected with regard to the intended route of administration and standard pharmaceutical practice.

The agents, medicaments and pharmaceutical compositions of the invention can also be administered parenterally, for example, intravenously, intra-arterially, intraperitoneally, intrathecally, intraventricularly, intrasternally, intracranially, intramuscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Medicaments and pharmaceutical compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The medicaments and compositions may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Generally, in humans, parenteral administration of the agents, medicaments and pharmaceutical compositions of the invention agents of the invention is the preferred route, being the most convenient.

For veterinary use, the agents, medicaments and pharmaceutical compositions of the invention are administered as a suitably acceptable formulation in accordance with normal veterinary practice and the veterinary surgeon will determine the dosing regimen and route of administration which will be most appropriate for a particular animal. Conveniently, the formulation is a pharmaceutical formulation. Advantageously, the formulation is a veterinary formulation.

### EXAMPLE 3

### Exemplary Pharmaceutical Formulations

Whilst it is possible for a agent of the invention to be administered alone, it is preferable to present it as a pharmaceutical formulation, together with one or more acceptable carriers. The carrier(s) must be “acceptable” in the sense of being compatible with the agent of the invention and not deleterious to the recipients thereof. Typically, the carriers will be water or saline which will be sterile and pyrogen-free.

The following examples illustrate medicaments and pharmaceutical compositions according to the invention in which the active ingredient is a nucleic acid or molecule of the invention.

### EXAMPLE 3A

### Injectable Formulation

| | | |
|---|---|---|
| Active ingredient | 0.20 | g |
| Sterile, pyrogen free phosphate buffer (pH 7.0) to | 10 | ml |

The active ingredient is dissolved in most of the phosphate buffer (35-40° C.), then made up to volume and filtered through a sterile micropore filter into a sterile 10 ml amber glass vial (type 1) and sealed with sterile closures and overseals.

### EXAMPLE 3B

### Intramuscular Injection

| | | |
|---|---|---|
| Active ingredient | 0.20 | g |
| Benzyl Alcohol | 0.10 | g |
| Glucofurol 75 ® | 1.45 | g |
| Water for Injection q.s. to | 3.00 | ml |

The active ingredient is dissolved in the glycofurol. The benzyl alcohol is then added and dissolved, and water added to 3 ml. The mixture is then filtered through a sterile micropore filter and sealed in sterile 3 ml glass vials (type 1).

### REFERENCES

Björke H, Andersson K. Measuring the affinity of a radioligand with its receptor using a rotating cell dish with in situ reference area. Appl Radiat Isot 2006; 64:32-7.
Bryan J N, Jia F, Mohsin H, Geethapriya S, Miller W H, Anderson C J, Henry C J, Lewis M R. Comparative uptakes and biodistributions of internalizing vs. noninternalizing copper-64 radioimmunoconjugates in cell and animal models of colon cancer. Nucl Med Biol 2005; 32:851-858.
Burvenich I, Schoonooghe S, Cornelissen B, Blanckaert P, Coene E, Cuvelier C, Mertens N, Slegers G. In vitro and in vivo targeting properties of iodine-123- or iodine-131-labeled monoclonal antibody 14C5 in a non-small cell lung cancer and colon carcinoma model. Clin Cancer Res 2005; 11:7288-96.
Derossi et al. (1998), Trends Cell Biol., 8, 84-87.
Fransson J, Ek S, Ellmark P, Söderlind E, Borrebaeck C A, Furebring C. Profiling of internalizing tumor-associated antigens on breast and pancreatic cancer cells by reversed genomics. Cancer Lett 2004; 208:235-42.
Fransson J, Tornberg U, Borrebaeck C A, Carlsson R, Frendéus B. Rapid induction of apoptosis in B cell lymphoma by functionally isolated human antibodies. Int J Cancer 2006 (in press)
Foehr E D, Lorente G, Kuo J, Ram R, Nikolich K, Urfer R. Targeting of the receptor protein tyrosine phosphatase beta with a monoclonal antibody delays tumor growth in a glioblastoma model. Cancer Res 2006; 66:2271-78.
Ginis I, Mentzer S J, Li X, Faller D V. Characterization of a hypoxia-responsive adhesion molecule for leukocytes on human endothelial cells. J Immunol 1995; 155:802-10.
Hanahan D, Weinberg R A. The hallmarks of cancer. Cell 2000; 100:57-70.
Lees-Miller S P, Meek K. Repair of DNA double strand breaks by non-homologous end joining. Biochimie 2003; 85:1161-73.
Lieber M R, Ma Y, Pannicke U, Schwarz K. Mechanism and regulation of human nonhomologous DNA end-joining. Nat Rev Mol Cell Biol 2003; 4:712-20.
Liu B, Conrad F, Cooperberg M R, Kirpotin D B, Marks J D. Mapping tumor epitope space by direct selection of single-chain Fv antibody libraries on prostate cancer cells. Cancer Res 2004; 64:704-710.
Martinez J J, Seveau S, Veiga E, Matsuyama S, Cossart P. Ku70, a component of DNA-dependent protein kinase, is a mammalian receptor for Rickettsia conorii. Cell 2005; 123:1013-23.
Monferran S, Muller C, Mourey L, Frit P, Salles B. The membrane-associated form of the DNA repair protein is involved in cell adhesion to fibronectin. J Mol Biol 2004; 337:503-11.
Monferran S, Paupert J, Dauvillier S, Salles B, Muller C. The membrane form of the DNA repair protein Ku interacts at the cell surface with metalloproteinase 9. EMBO J. 2004; 23:3758-68.
Muller C, Paupert J, Monferran S, Salles B. The double life of the Ku protein: facing the DNA breaks and the extracellular environment. Cell Cycle 2005; 4:438-441.
Norderhaug L, Olafsen T, Michaelsen T E, Sandlie I. Versatile vectors for transient and stable expression of recombinant antibody molecules in mammalian cells. J Immunol Methods 1997; 204:77-87.
Prabhakar B S, Allaway G P, Srinivasappa J, Notkins A L. Cell surface expression of the 70-kD component of Ku, a DNA-binding Nuclear Autoantigen. J Clin Invest 1990; 86:1301-5.
Samuelsson J, Dalevi D, Levander F, Rognvaldsson T. Modular, scriptable and automated analysis tools for high-throughput peptide mass fingerprinting. Bioinformatics 2004:20; 3628-35.
Sawada M, Sun W, Hayes P, Leskov K, Boothman D A, Matsuyama S. Ku70 suppresses the apoptotic translocation of Bax to mitochondria. Nat Cell Biol 2003; 5:320-9.
Shan D, Ledbetter J A, Press O W. Apoptosis of malignant human B cells by ligation of CD20 with monoclonal antibodies. Blood 1998; 91:1644-52.
Sundberg A L, Orlova A, Bruskin A, et al. [(111)In]Bz-DTPA-hEGF: Preparation and in vitro characterization of a potential anti-glioblastoma targeting agent. Cancer Biother Radiopharm 2003; 18:643-54.
Tai Y, Podar K, Kraeft S, et al. Translocation of Ku86/Ku70 to the multiple myeloma cell membrane: Functional implications. Exp Hematol 2002; 30:212-20.
Wu A M, Senter P D. Arming antibodies: prospects and challenges for immunoconjugates. Nat Biotechnol 2005; 23:1137-46.
Yang H M, Reisfeld R A. Doxorubicin conjugated with a monoclonal antibody directed to a human melanoma-associated proteoglycan suppresses the growth of established tumor xenografts in nude mice. Proc Natl Acad Sci USA 1988; 85:1189-93.
Zhang Y, Jeong Lee H, Boado R J, Pardridge W M. Receptor-mediated delivery of an antisense gene to human brain cancer cells. J Gene Med 2002; 4:183-94.

1. An agent comprising a binding moiety capable of selectively binding to Ku protein for use in medicine.

2. The agent according to claim 1 wherein the Ku protein is localised on the surface of a cell.

3. The agent according to claim 2 wherein the Ku protein is localised on the surface of a cancer cell.

4. The agent according to claim 1 wherein the agent is capable of inducing and/or increasing intracellular internalisation of the Ku protein and/or complex comprising the agent and Ku protein.

5. The agent according to claim 1 wherein the Ku protein is a mammalian protein.

6. The agent according to claim 5 wherein the Ku protein is a human protein.

7. The agent according to claim 6 wherein the Ku protein comprises the Ku-70 monomer and/or the Ku-80 monomer.

8. The agent according to claim 7 wherein the Ku protein is a heterodimer.

9. The agent according to claim 8 wherein the Ku protein is a Ku-70/80 heterodimer.

10. The agent according to claim 9 wherein the Ku70 protein comprises the polypeptide sequence of SEQ ID NO: 1 and/or is encoded by the polynucleotide sequence of SEQ ID NO: 3 and/or the Ku80 protein comprises the polypeptide sequence of SEQ ID NO:2 and/or is encoded by the polynucleotide sequence of SEQ ID NO:4.

11. The agent according to claim 9 further comprising a cytotoxic moiety.

12. The agent according to claim 11 wherein the cytotoxic moiety is directly and/or indirectly cytotoxic.

13. The agent according to claim 12 wherein the cytotoxic moiety is cytotoxic when intracellular.

14. The agent according to claim 13 wherein the cytotoxic moiety is not cytotoxic when extracellular.

15. The agent according to claim 11 wherein the cytotoxic moiety is a directly cytotoxic chemotherapeutic agent.

16. The agent according to claim 11 wherein the cytotoxic moiety is a directly cytotoxic polypeptide.

17. The agent according to claim 11 wherein the cytotoxic moiety is capable of converting a non-cytotoxic prodrug into a cytotoxic drug.

18. The agent according to claim 11 wherein the cytotoxic moiety is a radiosensitiser.

19. The agent according to claim 11 wherein the cytotoxic moiety is a nucleic acid molecule capable of converting a non-cytotoxic prodrug into a cytotoxic drug.

20. The agent according to claim 11 wherein the cytotoxic moiety is a directly cytotoxic nucleic acid molecule.

21. The agent according to claim 11 wherein the cytotoxic moiety is a nucleic acid molecule encoding a directly and/or indirectly cytotoxic polypeptide.

22. The agent according to claim 11 wherein the cytotoxic moiety is a nucleic acid molecule encoding a therapeutic polypeptide.

23. The agent according to claim 11 wherein the cytotoxic moiety comprises a radioactive atom.

24. The agent according to claim 23 wherein the radioactive atom is selected from the group comprising: phosphorous-32; iodine-125; iodine-131; indium-111; rhenium-186; rhenium-188; and yttrium-90.

25. The agent according to claim 24 further comprising a readily detectable moiety.

26. The agent according to claim 25 wherein the readily detectable moiety comprises a radioactive atom.

27. The agent according to claim 26 wherein the radioactive atom is technetium-99m or iodine-123.

28. The agent according to claim 25 wherein the readily detectable moiety is selected from the group comprising: iodine-123; iodine-131; indium-111; fluorine-19; carbon-13; nitrogen-15; oxygen-17; gadolinium; manganese; and iron.

29. The agent according to claim 28 further comprising a moiety capable of selectively binding to a directly or indirectly cytotoxic moiety.

30. The agent according to claim 28 further comprising a moiety capable of selectively binding to a readily detectable moiety.

31. The agent according to claim 28 wherein the binding moiety and the cytotoxic moiety are polypeptides which are fused to one another.

32. The agent according to claim 31 wherein the binding moiety comprises a peptide and/or a polypeptide.

33. The agent according to claim 32 wherein the binding moiety comprises a polypeptide sequence selected from the group comprising: SEQ ID NO:5; SEQ ID NO:6; SEQ ID NO:7; SEQ ID NO:8; SEQ ID NO:9; SEQ ID NO:10; and SEQ ID NO:11.

34. The agent according to claim 32 wherein the binding moiety comprises an antibody or a fragment thereof.

35. The agent according to claim 34 wherein the antibody or fragment thereof is an scFv or Fab.

36. The agent according to claim 35 wherein the scFv or Fab comprises the polypeptide sequence of SEQ ID NO: 11.

37.-41. (canceled)

42. A pharmaceutical composition comprising a therapeutically effective amount of an agent and a pharmaceutically-acceptable carrier, said agent comprising a binding moiety capable of selectively binding to Ku protein.

43. (canceled)

44. A method for treating cancer in an individual comprising the step of administering to the individual an effective amount of an agent, said agent comprising a binding moiety capable of selectively binding to Ku protein.

45. A method for identifying an individual having cancer cells potentially susceptible to treatment using an agent as, said agent comprising a binding moiety capable of selectively binding to Ku protein, the method comprising the steps of:
a) providing a sample comprising one or more cancer cell from the individual to be tested;
b) combining the sample with the agent;
c) determining binding of the agent to Ku protein localised on the surface of the one or more cancer cells, and subsequent intracellular internalisation of the Ku protein; and
d) identifying an individual having cancer cells potentially susceptible to treatment in the event that the agent induces and/or promotes intracellular internalisation of Ku protein localised on the surface of the one or more cancer cells.

46. A method for identifying an agent capable of selectively binding to Ku protein localised on the surface of a cell and inducing and/or increasing intracellular internalisation of the Ku protein comprising the steps of:
a) providing a sample comprising Ku protein localised on the surface of one or more cell;
b) combining the sample with an agent to be tested;
c) determining whether the agent binds to Ku protein localised on the surface of the one or more cell, and whether Ku protein is subsequently internalised; and
d) identifying an agent in the event that the agent is capable of selectively binding to Ku protein localised on the surface of a cell and inducing and/or increasing intracellular internalisation of the Ku protein.

47. The method according to claim 44 further comprising the step of:
e) synthesising and/or isolating the agent identified in step (d).

48. The method according to claim 47 further comprising the step of formulating the agent identified in step (d) and/or synthesised and/or isolated in step (e) into a pharmaceutical composition.

49. A nucleic acid molecule encoding an agent, said agent comprising a binding moiety capable of selectively binding to Ku protein.

50. An expression vector comprising a nucleic acid molecule according to claim 49.

51. A recombinant host cell comprising a nucleic acid molecule according to claim 49.

52. A recombinant host cell according to claim 51 wherein the host cell is a bacterial cell.

53. A recombinant host cell according to claim 52 wherein the host cell is a mammalian cell.

54. A method of producing an agent comprising culturing a host cell according to claim 51.

55. A kit of parts comprising an agent according to claim 17 and a relatively non-toxic prodrug.

56.-66. (canceled)


SEQUENCE LISTING (11 sequences)

SEQ ID NO: 1 - protein, 609 aa, Homo sapiens
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 2 - protein, 732 aa, Homo sapiens
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISLQFFLPFSLGKEDGSGDRGDGPFRLGGHGPSFPLKGITEQQKEGLEIVKMVMISLEGEDGLDEIYSFSESLRKLCVFKKIERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAKKLKTEQGGAHFSVSSLAEGSVTSVGSVNPAENFRVLVKQKKASFEEASNQLINHIEQFLDTNETPYFMKSIDCIRAFREEAIKFSEEQRFNNFLKALQEKVEIKQLNHFWEIVVQDGITLITKEEASGSSVTAEEAKKFLAPKDKPSGDTAAVFEEGGDVDDLLDMI

SEQ ID NO: 3 - DNA, 2156 bp, Homo sapiens
gcgcatgcgtggattgtcgtcttctgtccaagttggtcgcttccctgcgccaaagtgagcagtagccaacatgtcagggtgggagtcatattacaaaaccgagggcgatgaagaagcagaggaagaacaagaagagaaccttgaagcaagtggagactataaatattcaggaagagatagtttgatttttttggttgatgcctccaaggctatgtttgaatctcagagtgaagatgagttgacaccttttgacatgagcatccagtgtatccaaagtgtgtacatcagtaagatcataagcagtgatcgagatctcttggctgtggtgttctatggtaccgagaaagacaaaaattcagtgaattttaaaaatatttacgtcttacaggagctggataatccaggtgcaaaacgaattctagagcttgaccagtttaaggggcagcagggacaaaaacgtttccaagacatgatgggccacggatctgactactcactcagtgaagtgctgtgggtctgtgccaacctctttagtgatgtccaattcaagatgagtcataagaggatcatgctgttcaccaatgaagacaacccccatggcaatgacagtgccaaagccagccgggccaggaccaaagccggtgatctccgagatacaggcatcttccttgacttgatgcacctgaagaaacctgggggctttgacatatccttgttctacagagatatcatcagcatagcagaggatgaggacctcagggttcactttgaggaatccagcaagctagaagacctgttgcggaaggttcgcgccaaggagaccaggaagcgagcactcagcaggttaaagctgaagctcaacaaagatatagtgatctctgtgggcatttataatctggtccagaaggctctcaagcctcctccaataaagctctatcgggaaacaaatgaaccagtgaaaaccaagacccggacctttaatacaagtacaggcggtttgcttctgcctagcgataccaagaggtctcagatctatgggagtcgtcagattatactggagaaagaggaaacagaagagctaaaacggtttgatgatccaggtttgatgctcatgggtttcaagccgttggtactgctgaagaaacaccattacctgaggccctccctgttcgtgtacccagaggagtcgctggtgattgggagctcaaccctgttcagtgctctgctcatcaagtgtctggagaaggaggttgcagcattgtgcagatacacaccccgcaggaacatccctccttattttgtggctttggtgccacaggaagaagagttggatgaccagaaaattcaggtgactcctccaggcttccagctggtctttttaccctttgctgatgataaaaggaagatgccctttactgaaaaaatcatggcaactccagagcaggtgggcaagatgaaggctatcgttgagaagcttcgcttcacatacagaagtgacagctttgagaaccccgtgctgcagcagcacttcaggaacctggaggccttggccttggatttgatggagccggaacaagcagtggacctgacattgcccaaggttgaagcaatgaataaaagactgggctccttggtggatgagtttaaggagcttgtttacccaccagattacaatcctgaagggaaagttaccaagagaaaacacgataatgaaggttctggaagcaaaaggcccaaggtggagtattcagaagaggagctgaagacccacatcagcaagggtacgctgggcaagttcactgtgcccatgctgaaagaggcctgccgggcttacgggctgaagagtgggctgaagaagcaggagctgctggaagccctcaccaagcacttccaggactgaccagaggccgcgcgtccagctgcccttccgcagtgtggccaggctgcctggccttgtcctcagccagttaaaatgtgtttctcctgagctaggaagagtctacccgacataagtcgagggactttatgtttttgaggctttctgttgccatggtgatggtgtagccctcccactttgctgttccttactttactgcctgaataaagagccctaagtttgtactatatactgttaaaaaaaaaaaaaaaaaaaaaaa

SEQ ID NO: 4 - DNA, 3310 bp, Homo sapiens
ggcgggcgaccaaagcgcctgaggaccggcaacatggtgcggtcggggaataaggcagctgttgtgctgtgtatggacgtgggctttaccatgagtaactccattcctggtatagaatccccatttgaacaagcaaagaaggtgataaccatgtttgtacagcgacaggtgtttgctgagaacaaggatgagattgctttagtcctgtttggtacagatggcactgacaatcccctttctggtggggatcagtatcagaacatcacagtgcacagacatctgatgctaccagattttgatttgctggaggacattgaaagcaaaatccaaccaggttctcaacaggctgacttcctggatgcactaatcgtgagcatggatgtgattcaacatgaaacaataggaaagaagtttgagaagaggcatattgaaatattcactgacctcagcagccgattcagcaaaagtcagctggatattataattcatagcttgaagaaatgtgacatctccctgcaattcttcttgcctttctcacttggcaaggaagatggaagtggggacagaggagatggcccctttcgcttaggtggccatgggccttcctttccactaaaaggaattaccgaacagcaaaaagaaggtcttgagatagtgaaaatggtgatgatatctttagaaggtgaagatgggttggatgaaatttattcattcagtgagagtctgagaaaactgtgcgtcttcaagaaaattgagaggcattccattcactggccctgccgactgaccattggctccaatttgtctataaggattgcagcctataaatcgattctacaggagagagttaaaaagacttggacagttgtggatgcaaaaaccctaaaaaaagaagatatacaaaaagaaacagtttattgcttaaatgatgatgatgaaactgaagttttaaaagaggatattattcaagggttccgctatggaagtgatatagttcctttctctaaagtggatgaggaacaaatgaaatataaatcggaggggaagtgcttctctgttttgggattttgtaaatcttctcaggttcagagaagattcttcatgggaaatcaagttctaaaggtctttgcagcaagagatgatgaggcagctgcagttgcactttcctccctgattcatgctttggatgacttagacatggtggccatagttcgatatgcttatgacaaaagagctaatcctcaagtcggcgtggcttttcctcatatcaagcataactatgagtgtttagtgtatgtgcagctgcctttcatggaagacttgcggcaatacatgttttcatccttgaaaaacagtaagaaatatgctcccaccgaggcacagttgaatgctgttgatgctttgattgactccatgagcttggcaaagaaagatgagaagacagacacccttgaagacttgtttccaaccaccaaaatcccaaatcctcgatttcagagattatttcagtgtctgctgcacagagctttacatccccgggagcctctacccccaattcagcagcatatttggaatatgctgaatcctcccgctgaggtgacaacaaaaagtcagattcctctctctaaaataaagaccctttttcctctgattgaagccaagaaaaaggatcaagtgactgctcaggaaattttccaagacaaccatgaagatggacctacagctaaaaaattaaagactgagcaagggggagcccacttcagcgtctccagtctggctgaaggcagtgtcacctctgttggaagtgtgaatcctgctgaaaacttccgtgttctagtgaaacagaagaaggccagctttgaggaagcgagtaaccagctcataaatcacatcgaacagtttttggatactaatgaaacaccgtattttatgaagagcatagactgcatccgagccttccgggaagaagccattaagttttcagaagagcagcgctttaacaacttcctgaaagcccttcaagagaaagtggaaattaaacaattaaatcatttctgggaaattgttgtccaggatggaattactctgatcaccaaagaggaagcctctggaagttctgtcacagctgaggaagccaaaaagtttctggcccccaaagacaaaccaagtggagacacagcagctgtatttgaagaaggtggtgatgtggacgatttattggacatgatataggtcgtggatgtatggggaatctaagagagctgccatcgctgtgatgctgggagttctaacaaaacaagttggatgcggccattcaaggggagccaaaatctcaagaaattcccagcaggttacctggaggcggatcatctaattctctgtggaatgaatacacacatatatattacaagggataatttagaccccatacaagtttataaagagtcattgttattttctggttggtgtattattttttctgtggtcttactgatctttgtatattacatacatgctttgaagtttctggaaagtagatcttttcttgacctagtatatcagtgacagttgcagcccttgtgatgtgattagtgtctcatgtggaaccatggcatggttattgatgagtttcttaaccctttccagagtcctcctttgcctgatcctccaacagctgtcacaacttgtgttgagcaagcagtagcatttgcttcctcccaacaagcagctgggttaggaaaaccatgggtaaggacggactcacttctctttttagttgaggccttctagttaccacattactctgcctctgtatataggtggttttctttaagtggggtgggaaggggagcacaatttcccttcatactccttttaagcagtgagttatggtggtggtctcatgaagaaaagaccttttggcccaatctctgccatatcagtgaacctttagaaactcaaaaactgagaaatttacttcagtagttagaattatatcacttcactgttctctacttgcaagcctcaaagagagaaagtttcgttatattaaaacacttaggtaacttttcggtctttcccatttctacctaagtcagctttcatctttgtggatggtgtctcctttactaaataagaaaataacaaagcccttattctctttttttcttgtcctcattcttgccttgagttccagttcctctttggtgtacagacttcttggtacccagtcacctctgtcttcagcaccctcataagtcgtcactaatacacagttttgtacatgtaacattaaaggcataaatgactc

SEQ ID NO: 5 - protein, 7 aa, Unknown
  INCA-X VH CDR1 polypeptide
FSRYGMH

SEQ ID NO: 6 - protein, 17 aa, Unknown
  INCA-X VH CDR2 polypeptide
RINSDGSEAKYADSVKG

SEQ ID NO: 7 - protein, 8 aa, Unknown
  INCA-X VH CDR3 polypeptide
HSYNGMDV

SEQ ID NO: 8 - protein, 13 aa, Unknown
  INCA-X VL CDR1 polypeptide
SGSSSNIGSHYAY

SEQ ID NO: 9 - protein, 7 aa, Unknown
  INCA-X VL CDR2 polypeptide
SNRQRPS

SEQ ID NO: 10 - protein, 11 aa, Unknown
  INCA-X VL CDR3 polypeptide
AAWDDRLSGPV

SEQ ID NO: 11 - protein, 243 aa, Unknown
  INCA-X polypeptide
EVQLLESGGGLVQPGGSLRLSCAASGFTFSRYGMHWVRQAPGKGLEWVSRINSDGSEAKYADSVKGRFTISRDNSKNTLYLQMNSLRAEDTAVYYCARHSYNGMDVWGQGTLVTVSSGGGGSGGGGSGGGGSQSVLTQPPSASGTPGQRVTISCSGSSSNIGSHYAYWYQQLPGTAPKLLIYSNRQRPSGVPDRFSGSKSGTSASLAISGLRSEDEADYYCAAWDDRLSGPVFGGGTKLTVLG